(19) United States
(12) Patent Application Publication
(10) Pub. No.: US 2026/0279486 A1
(43) Pub. Date: Sep. 17, 2026

(54) METHODS AND COMPOSITIONS FOR FERTILITY ENHANCEMENT AND GENETIC OPTIMIZATION ACROSS SPECIES

(71) Applicants: Mena ABDELSAYED, Philadelphia, PA (US); Sabine GRIMES, Toronto (CA); Ann MEYER, Toronto (CA)

(72) Inventors: Mena ABDELSAYED, Philadelphia, PA (US); Sabine GRIMES, Toronto (CA); Ann MEYER, Toronto (CA)

(21) Appl. No.: 19/566,786

(22) Filed: Mar. 13, 2026

Related U.S. Application Data
(60) Provisional application No. 63/773,008, filed on Mar. 17, 2025.

(51) Int. Cl. G16B 10/00; G16B 20/40; G16B 25/10; G16B 30/10; G16B 50/30; G16H 15/00; G16H 50/30

(52) U.S. Cl. CPC G16B 10/00; G16B 20/40; G16B 25/10; G16B 30/10; G16B 50/30; G16H 15/00; G16H 50/30

(57) ABSTRACT

This disclosure relates to a system and method for fertility enhancement and genetic optimization across humans, animals, and plants. The system and method described herein utilize specialized hardware acceleration such as, for example, field-programmable gate arrays (FPGAs) to enable efficient genetic sequence analysis, and integrates artificial intelligence algorithms to assess genetic compatibility between donors and recipients. By analyzing genetic markers associated with dominant and recessive traits, the system optimizes gamete selection, minimizes hereditary risks, and enhances desired phenotypic outcomes. The system and methods incorporate a comprehensive genetic database architecture with optimized data structures for rapid retrieval and analysis, enabling the assessment of genetic compatibility with improved computational efficiency. An AI-based decision framework prioritizes traits such as disease resistance, productivity, and environmental adaptability while mitigating recessive genetic risks.

### CROSS-REFERENCE TO RELATED APPLICATIONS

This application claims the benefit of and priority to U.S. Provisional Patent Application No. 63/773,008, filed on Mar. 17, 2025, the entire contents of which are hereby incorporated by reference herein in their entirety.

### FIELD

The present invention relates to genetic optimization systems and methods, particularly to an integrated hardware-software platform that enhances fertility outcomes and optimizes genetic traits across humans, animals, and plants. The invention leverages specialized hardware acceleration techniques, such as, for example, field-programmable gate arrays (FPGAs), combined with advanced bioinformatics and artificial intelligence algorithms, to improve computational efficiency in genetic sequence analysis, enabling more effective gamete selection, optimized fertilization outcomes, and enhanced phenotypic traits through comprehensive genetic analysis.

### BACKGROUND

Citation or identification of any document in this application is not an admission that such document is available as prior art to the present invention. The field of genetic optimization has historically faced significant challenges in reproductive and agricultural sciences. Traditional breeding approaches, which relied primarily on phenotypic traits or limited genetic knowledge, often resulted in suboptimal outcomes, including low fertility rates, hereditary disease propagation, and poor agricultural yields. While recent advances in genetic profiling technologies have enabled a deeper understanding of genetic markers and their associations with traits such as disease resistance and productivity, existing solutions face substantial technical limitations. Current systems struggle with computational efficiency when processing large-scale genetic data and lack integrated frameworks for cross-species adaptation. Furthermore, available platforms typically fail to effectively combine artificial intelligence-based decision-making with hardware acceleration techniques, resulting in suboptimal performance and scalability limitations.

While conventional dairy breeding systems rely on crossbreeding strategies utilizing static, pre-computed selection indices (e.g., NM$, CM$) or generalized GEBVs (see, e.g., U.S. Pat. Nos. 10,159,226 and 10,975,351, incorporated herein by reference), such existing systems fail to account for pair-specific dominance interactions, do not propagate genotype uncertainty through probabilistic simulation, and do not employ hardware-accelerated combinatorial optimization for cohort-level mating assignments.

### SUMMARY

The present disclosure provides for solutions that address the technical challenges in genetic optimization through a novel hardware-software system that significantly improves computational efficiency in genetic sequence analysis. The invention stems from the finding that implementing specialized hardware acceleration, such as, for example, field-programmable gate arrays (FPGAs), configured for genetic sequence alignment algorithms substantially enhances processing speed and enables more comprehensive genetic compatibility assessment.

In an embodiment, a system for genetic profile analysis and compatibility assessment comprises a specialized hardware acceleration unit with field-programmable gate arrays (FPGAs) configured to implement sequence alignment algorithms to process genetic sequence data. The system includes a genetic profile analyzer that has a data acquisition module configured to receive genetic sequence data, a hardware-accelerated sequence alignment processor configured to identify genetic markers within the received genetic sequence data using parallel processing architecture, and a trait analyzer configured to classify identified genetic markers as dominant or recessive traits based on predefined genetic inheritance models. The system further includes a genetic compatibility assessment engine comprising a match scoring module configured to calculate genetic compatibility scores using weighted comparison of identified dominant and recessive traits, and a risk assessment module configured to evaluate genetic compatibility risks using statistical probability models. A database management system is configured to store genetic profile data using data structures optimized for genetic sequence retrieval and maintain genetic marker indices optimized for accelerated data retrieval. The system implements a defined processing pipeline for transferring genetic data between modules and utilizes the field-programmable gate arrays (FPGAs) to accelerate genetic sequence alignment operations through parallel computation of sequence matching matrices.

In an embodiment, the genetic compatibility assessment engine is further configured to identify complementary genetic profiles by evaluating how genetic markers from different subjects would function together in offspring, calculate an optimal mating distance score that balances genetic diversity benefits against incompatibility risks, and generate a ranked list of genetic matches based on the calculated genetic compatibility scores and optimal mating distance scores.

In an embodiment, the trait analyzer is further configured to classify genetic markers according to inheritance patterns including dominant, recessive, co-dominant, and polygenic traits, identify carrier status for recessive disease alleles, and evaluate immune system compatibility markers including Human Leukocyte Antigen (HLA) genes in humans or corresponding markers in non-human species.

In an embodiment, the field-programmable gate arrays (FPGAs) are configured to implement an array architecture specifically optimized for executing Smith-Waterman or Needleman-Wunsch sequence alignment algorithms, incorporate dedicated logic blocks for parallel computation of nucleotide scoring matrices, and utilize specialized memory access patterns that minimize data transfer latency during genetic sequence comparison operations.

In an embodiment, the system is configured to operate across multiple species through a layer of abstraction that translates species-specific genetic concepts into standardized representations, species-specific modules that address unique genetic characteristics of humans, animals, or plants, and cross-species orthologous gene mapping to facilitate knowledge transfer across application domains.

In an embodiment, for human applications, the system prioritizes health-related genetic compatibility assessment and hereditary disease risk minimization; for animal applications, the system emphasizes genetic compatibility assessment, hereditary disease risk minimization, genetic diversity management, and trait optimization for health, productivity or conservation; and for plant applications, the system focuses on yield improvement, disease resistance, and environmental adaptability. In an embodiment, a method for genetic profile analysis and compatibility assessment comprises receiving genetic sequence data from multiple subjects, processing the genetic sequence data using field-programmable gate arrays (FPGAs) configured to accelerate sequence alignment operations through parallel computation, identifying genetic markers within the processed genetic sequence data, classifying the identified genetic markers as dominant or recessive traits based on predefined genetic inheritance models, calculating genetic compatibility scores between subjects using weighted comparison of the classified genetic markers, evaluating genetic compatibility risks using statistical probability models, and generating a ranked list of genetically compatible matches based on the calculated compatibility scores and evaluated risks.

In an embodiment, the method further comprises analyzing DNA matching patterns to identify complementary genetic profiles that would function optimally together in offspring, calculating an optimal mating distance that balances genetic diversity benefits with incompatibility risks, and predicting offspring trait expressions using statistical probability models based on parental genetic markers.

In an embodiment, calculating genetic compatibility scores comprises assigning different weights to genetic markers based on their impact on traits of interest, evaluating compatibility at critical genetic loci including immune system genes, calculating a genetic distance measure between subjects, and integrating these factors into a composite compatibility score.

In an embodiment, processing the genetic sequence data using field-programmable gate arrays (FPGAs) comprises implementing hardware-optimized versions of sequence alignment algorithms, utilizing an array architecture for parallel computation of alignment matrices, and employing specialized memory access patterns that optimize cache utilization and minimize memory bandwidth requirements.

In an embodiment, a method for evaluating genetic compatibility between potential mates comprises obtaining genetic profiles from potential mates through DNA sequencing, analyzing the genetic profiles using hardware-accelerated sequence alignment to identify genetic markers, classifying the genetic markers according to inheritance patterns and associated traits, screening for recessive disease mutations and calculating carrier risk probabilities, calculating genetic compatibility scores between a subject and potential mates using weighted comparison of genetic markers, determining an optimal genetic distance between potential mates that balances heterosis benefits with genetic incompatibility risks, and generating a prioritized list of genetically compatible mates based on the calculated scores, recessive mutation screening, and optimal genetic distance.

In an embodiment, the method further comprises evaluating compatibility at specific genetic loci, including Human Leukocyte Antigen (HLA) genes or equivalent immune system markers, calculating statistical probabilities for offspring trait expression based on potential mate combinations, predicting the likelihood of hereditary disease transmission in offspring, and generating detailed compatibility reports that present findings in an actionable format.

In an embodiment, a non-transitory computer-readable medium stores instructions that, when executed by a processor, cause the processor to perform a method for genetic profile analysis and compatibility assessment, the method comprising receiving genetic sequence data from multiple subjects, processing the genetic sequence data using hardware-accelerated sequence alignment operations, identifying genetic markers within the processed genetic sequence data, classifying the identified genetic markers as dominant or recessive traits, calculating genetic compatibility scores between subjects using weighted comparison of the classified genetic markers, evaluating genetic compatibility risks using statistical probability models, and generating a ranked list of genetically compatible matches based on the calculated compatibility scores and evaluated risks.

Applications include human fertility treatments, livestock breeding programs, conservation efforts, and agricultural optimization, offering significant improvements in reproductive outcomes, genetic diversity, and trait selection across multiple industries.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description, given by way of example, but not intended to limit the invention solely to the specific embodiments described, may best be understood in conjunction with the accompanying drawings wherein:

FIG. 1 illustrates a block diagram illustrating the overall system architecture of the genetic optimization platform, showing the relationship between hardware acceleration components, software modules, and data flow paths.

FIG. 2 depicts a schematic diagram showing the FPGA configuration for genetic sequence alignment, illustrating the systolic array architecture and parallel processing elements optimized for executing sequence alignment algorithms.

FIG. 3 depicts a flowchart depicting a decision-tree algorithm for genetic matching, illustrating the process flow from genetic profile input through compatibility assessment to final match ranking.

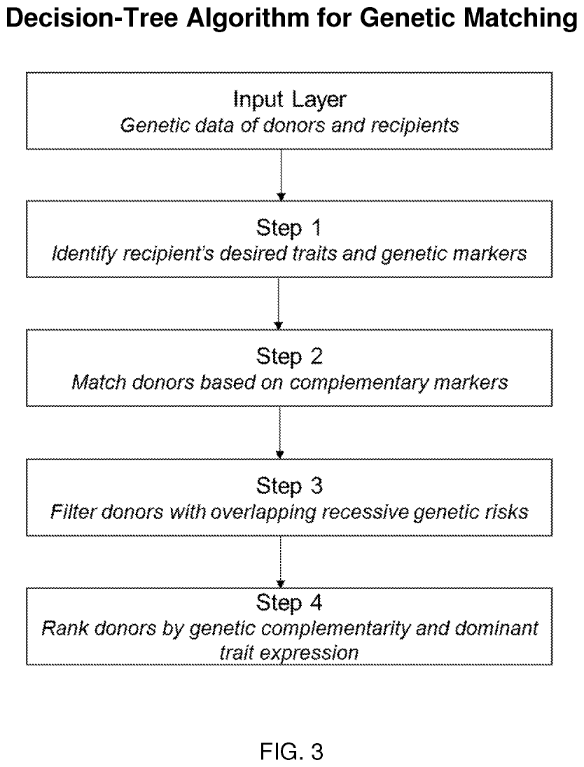

FIG. 4 illustrates pseudocode for a genetic matching algorithm, showing the key computational steps for filtering complementary genetic traits and ranking potential donors based on dominance scores.

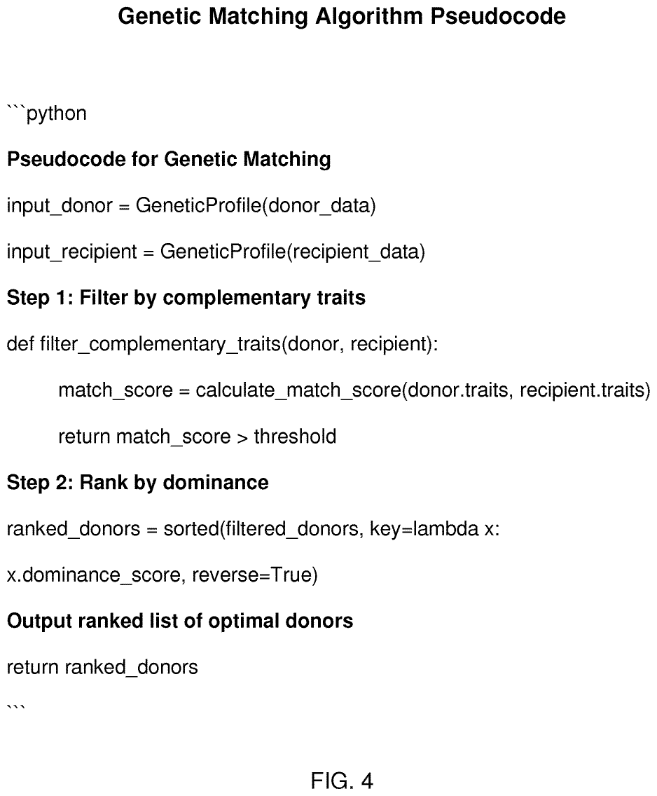

FIG. 5 depicts a diagram showing the database architecture for genetic marker storage and retrieval, illustrating the data structures and indexing mechanisms optimized for efficient genetic data management.

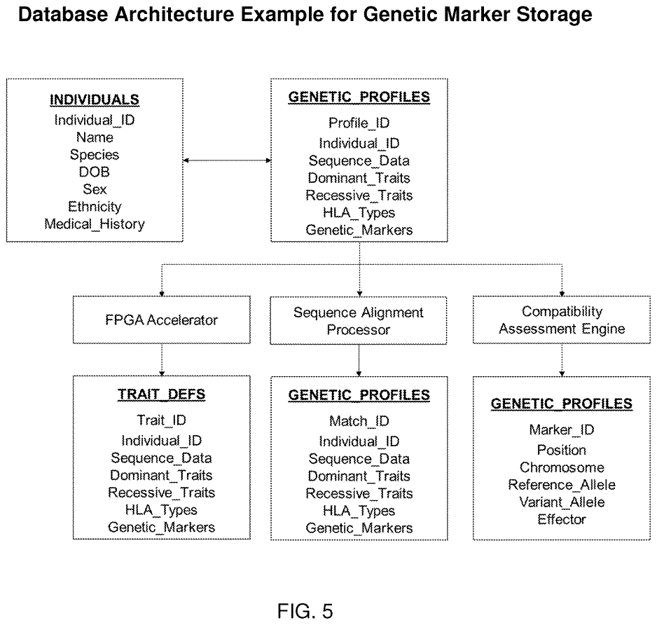

FIG. 6 depicts a example Monte Carlo simulation loop for genotype and parameter sampling. An example probabilistic simulation workflow is depicted in which parental genotype data and posterior parameter distributions are used to iteratively sample offspring genotypes and model parameters (for example, additive effects, dominance effects, penetrance terms, logistic coefficients, and Markov transition probabilities), compute simulated offspring outcomes (for example, trait values, disease risk, threshold events, and state transitions), aggregate posterior predictive summaries across simulation iterations (such as means, variances, quantiles, threshold probabilities, tail-risk metrics, and deleterious genotype probabilities), and generate compatibility scores and mating recommendations.

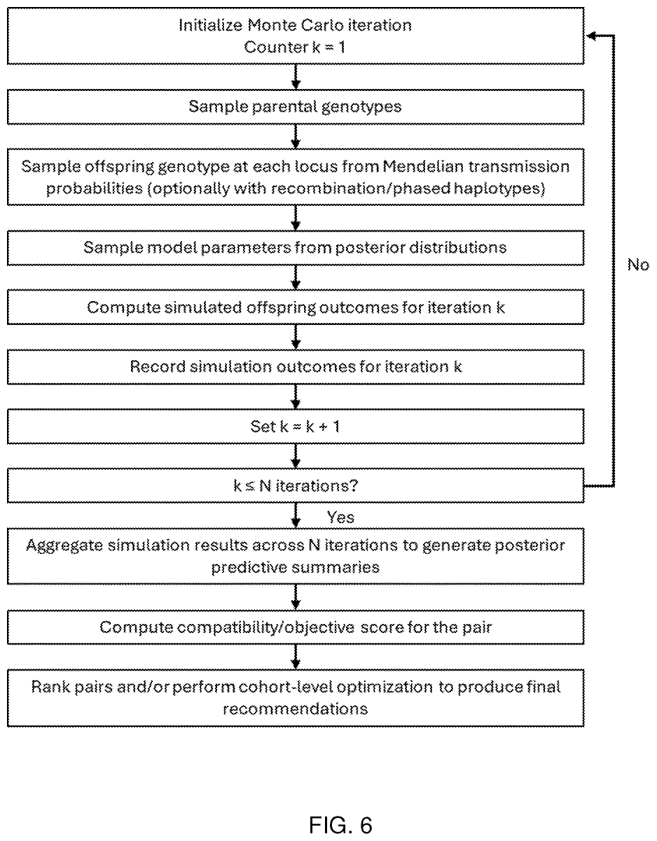

FIG. 7 illustrates an example complementarity index module and objective function evaluation. An example computational workflow is depicted in which parental genotype data, offspring genotype probability distributions, and posterior model parameter distributions are used to compute a complementarity index. The complementarity index is evaluated by an objective function that incorporates expected trait values, risk penalties, and user-defined or algorithm-tuned weights to produce compatibility scores used for pair ranking.

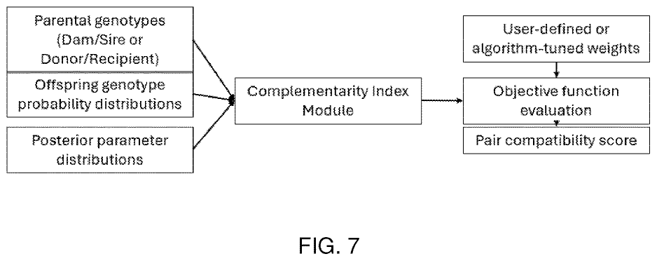

### DETAILED DESCRIPTION

The present disclosure stems from the finding that implementing specialized hardware acceleration, such as, for example, field-programmable gate arrays (FPGAs), for genetic sequence analysis significantly enhances computational efficiency in genetic compatibility assessment. Processing whole-genome sequence alignments across N×M candidate pairings, particularly when propagating genotype uncertainty through 100,000+ Monte Carlo simulations per pairing, exceeds the practical computational capacity of general-purpose processors. Conventional software-based systems require days or weeks of processing time to optimize a single breeding cohort. The present disclosure provides a specific technical solution to this computational bottleneck by utilizing custom Field-Programmable Gate Array (FPGA) logic circuits that implement genomic hashing, affine gap penalty handling, and probability matrix multiplications directly in hardware, reducing cohort-level optimization time from days to hours and providing a tangible improvement to the functioning of the computer itself.

When combined with optimized data structures and machine learning algorithms, this approach enables the precise identification of complementary genetic markers across diverse species. Furthermore, the disclosure demonstrates that applying statistical probability models to evaluate genetic compatibility leads to improved prediction of genetic outcomes, allowing for targeted optimization of fertility outcomes and genetic trait expression. By integrating these technological advances into a cohesive system architecture, substantial improvements in genetic profile analysis, compatibility assessment, and trait optimization can be achieved across human, animal, and plant applications.

Furthermore, the system transforms raw genetic sequence data, a physical input derived from biological samples, through a series of specific, hardware-accelerated computational steps into a concrete, actionable output: ranked mating recommendations or cohort-level breeding assignments that are subsequently physically executed through insemination, embryo transfer, or cross-pollination. The physical execution of these recommendations results in the conception and gestation of genetically optimized offspring, representing a tangible transformation of matter that goes well beyond the mere manipulation of abstract data. The system's integration of specialized hardware, probabilistic statistical models, and species-specific biological knowledge into a unified platform produces results that could not be achieved by any one component alone.

In the context of animal husbandry and agriculture, conventional crossbreeding strategies intended to capture heterosis, or hybrid vigor, face significant challenges in maintaining these advantages in subsequent generations. While an initial F1 hybrid animal is a uniform composite receiving exactly 50% of its genome from each founder breed, generating replacement progeny from these F1 subjects introduces complications due to Mendelian independent assortment and chromosomal recombination. The haploid gametes produced by an F1 hybrid will contain highly variable, unpredictable combinations of the founder genomes, resulting in a normal distribution centered at a 50:50 ratio but approaching 100% of either founder breed at the tails. Consequently, conventional mating of F1 hybrids often results in progeny with significant phenotypic dis-uniformity and a loss of hybrid vigor.

Furthermore, while advanced reproductive technologies such as in vitro fertilization (IVF), embryo transfer (ET), and somatic cell nuclear transfer (SCNT) are utilized in breeding, they are often deemed too expensive or complex for widespread commercial populations. An integrated, computationally efficient platform capable of probabilistically optimizing crosses and managing the complex genetic interactions is required to sustainably produce and maintain high-performing hybrid populations.

Before the present compositions and methods are described, it is to be understood that this invention is not limited to the particular compositions, methods, and experimental conditions described, as such compositions, methods, and conditions may vary. It is also to be understood that the terminology used herein is for purposes of describing particular embodiments only and is not intended to be limiting since the scope of the present invention will be limited only to the appended claims.

As used in this specification and the appended claims, the singular forms “a,” “an,” and “the” refer to one or to more than one (e.g., to at least one) of the grammatical object of the article and include plural references unless the context clearly dictates otherwise. Thus, for example, references to “the method” include one or more methods and/or steps of the type described herein, which will become apparent to those persons skilled in the art upon reading this disclosure and so forth. By way of example, “a genetic marker” means one genetic marker or more than one genetic marker.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skilled in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used herein, the term “genetic profile” refers to a comprehensive or partial set of genetic information derived from a subject's genetic material, including but not limited to complete genome sequences, exome sequences, targeted gene panels, identified genetic markers, variants, structural variations, and their classifications as dominant or recessive traits. A genetic profile may comprise single nucleotide polymorphisms (SNPs), copy number variations (CNVs), insertions, deletions, inversions, translocations, epigenetic modifications, and other genetic features that characterize a subject organism.

As used herein, the term “genetic marker” refers to a DNA sequence with a known physical location on a chromosome or a measurable variation in DNA that can be used to identify inherited traits, diseases, or other genetic characteristics. In an embodiment, genetic markers may include, but are not limited to, single nucleotide polymorphisms (SNPs), insertions, deletions, short tandem repeats (STRs), variable number tandem repeats (VNTRs), copy number variations (CNVs), structural variants (SVs), chromosomal rearrangements, epigenetic modifications such as DNA methylation patterns, histone modifications, and other identifiable genetic or epigenetic variations.

As used herein, the term “Locus” (loci plural) or “site” and their grammatical equivalents refer to a specific place or places on a chromosome where a gene, a genetic marker or a QTL is found.

As used herein, the term “quantitative trait locus (QTL)” refers to a contiguous section of DNA (e.g., a chromosome arm, a chromosome region, a nucleotide sequence, a gene) that is closely linked to a gene that underlies a trait of interest. “QTL mapping” involves the creation of a map of the genome using genetic or molecular markers, visible polymorphisms and allozymes, and determining the degree of association of a specific region on the genome to the inheritance of the trait of interest. The markers do not necessarily involve genes. QTL mapping results involve the degree of association of a stretch of DNA with a trait rather than pointing directly at the gene responsible for that trait. Statistical methods as described herein can be used to ascertain whether the degree of association is significant or not. A molecular marker is said to be “linked” to a gene or locus if the marker and the gene or locus have a greater association in inheritance than would be expected from independent assortment, i.e. the marker and the locus co-segregate in a segregating population and are located on the same chromosome. “Linkage” refers to the genetic distance of the marker to the locus or gene (or two loci or two markers to each other). The closer the linkage, the smaller the likelihood of a recombination event taking place, which separates the marker from the gene or locus. Genetic distance (map distance) is calculated from recombination frequencies and is expressed in centiMorgans (cM).

As used herein, the term “genetic optimization” refers to any process, method, or system designed to enhance genetic outcomes through the selection, combination, or modification of genetic material. Genetic optimization encompasses techniques for improving heritable traits, reducing disease risk, increasing genetic diversity, enhancing reproductive success, improving survivability, increasing productivity, or otherwise producing more favorable genetic configurations in offspring or subsequent generations. In an embodiment, genetic optimization may be applied to humans, animals, plants, or other organisms.

As used herein, the term “fertility enhancement” refers to any improvement in reproductive success, including but not limited to increased conception rates, improved embryo viability, reduced fetal loss, increased live birth rates, improved gamete quality, increased genetic compatibility between reproductive partners, optimized timing of reproduction, or any other factors that contribute to successful reproduction across species. In an embodiment, fertility enhancement may involve interventions at any stage of the reproductive process, from gamete formation to offspring development.

As used herein, the term “artificial intelligence” or “AI” refers to computational systems that can perform tasks that typically require human intelligence. This includes, but is not limited to, machine learning algorithms, neural networks, deep learning systems, decision trees, random forests, support vector machines, Bayesian networks, genetic algorithms, and other statistical or probabilistic models capable of data analysis, pattern recognition, decision making, or prediction without explicit programming for each specific task. In the context of genetic analysis, AI encompasses systems that can recognize patterns in genetic data, predict genetic outcomes, optimize genetic selection, and adapt their analytical approaches based on new data.

In some embodiments, this disclosure provides for methods which include training an artificial intelligence model with a first training data set to identify one or more compatible nucleotide sequences for genetic compatibility assessment (e.g., for purposes of breeding trait optimization, fertilization event, or progeny phenotype), wherein the artificial intelligence model is selected from a set of: a neural network model, a Bayesian network model, a support vector machine model, a k-nearest neighbors model, or a combination thereof. In some embodiments, the methods disclosed herein further comprise obtaining a first subject nucleotide sequencing data relating to a first subject or first subject's population; identifying using the trained artificial intelligence model and the first subject nucleotide sequencing data, a first set of one or more compatible nucleotide sequences for identifying genetic compatibility.

As used herein, the term “hardware acceleration” refers to the use of specialized hardware components to perform certain computational tasks more efficiently than general-purpose computing hardware. Hardware acceleration includes, but is not limited to, field-programmable gate arrays (FPGAs), graphics processing units (GPUs), application-specific integrated circuits (ASICs), tensor processing units (TPUs), digital signal processors (DSPs), and other current or future specialized computing architectures designed to accelerate specific computational tasks, particularly those related to genetic sequence alignment, comparison, and analysis.

As used herein, the term “systolic array” refers to a homogeneous network of tightly coupled data processing units (DPUs) or elements called cells or nodes, typically hardwired within an FPGA or ASIC, that independently compute partial results as data flows through them. In genetic sequence alignment, a systolic array maps the computational dependencies of dynamic programming matrices (e.g., Smith-Waterman scoring matrices) directly onto the hardware, allowing multiple nucleotide comparisons to execute in a single clock cycle.

As used herein, the term bitstream refers to a file containing the programming information that defines the hardware configuration and logic gate routing within an FPGA. Loading a custom bitstream physically transforms the generalized logic blocks of the FPGA into a specialized application-specific circuit dedicated to executing genomic hashing, sequence alignment, or probabilistic matrix multiplications.

As used herein, the term “complementary genetic markers” refers to combinations of genetic features across two or more genetic profiles that, when combined, produce favorable genetic outcomes. In an embodiment, complementary genetic markers may include dominant alleles that counterbalance recessive disease-causing alleles, genetic variants that together enhance desirable traits, or genetic configurations that minimize the expression of deleterious traits while maximizing the expression of beneficial traits. In an embodiment, complementary genetic markers may have either positive associations (enhancing desirable traits) or negative associations (reducing undesirable traits) when combined.

As used herein, the term “gamete” refers to any reproductive cell across all species that can fuse with another reproductive cell during fertilization to form a zygote. In animals, gametes include sperm and eggs (ova); in plants, gametes include pollen and ovules; in fungi, gametes include various specialized cells involved in sexual reproduction. Gametes typically contain a haploid set of chromosomes that combine with another haploid set during fertilization to produce a diploid zygote.

As used herein, the term “subject” refers to any organism that serves as a source of genetic material or as a target for genetic analysis or optimization. In an embodiment, subjects may include humans, non-human animals (including mammals, birds, reptiles, amphibians, fish, and invertebrates), plants (including agricultural crops, ornamental plants, trees, and wild plants), fungi, bacteria, or other organisms. In an embodiment, a subject may refer to a patient, an agricultural product, or an organism of conservation concern.

As used herein, the term “trait” refers to any distinguishable characteristic, feature, or property of an organism that may have a genetic basis. In an embodiment, traits may include phenotypic characteristics (such as physical appearance, behavior, or disease susceptibility), molecular features (such as protein structure, enzyme activity, or metabolic processes), physiological functions (such as immune response, hormone production, or reproductive capability), or any other attributes that can be influenced by genetic factors. In an embodiment, traits may be qualitative (discrete) or quantitative (continuous) and may be determined by single genes, multiple genes, environmental factors, or interactions between genes and environment.

As used herein, the term “database” refers to any organized collection of genetic data that allows for storage, retrieval, and analysis of information. In an embodiment, a database may be structured as a relational database, object-oriented database, graph database, key-value store, document store, or any other data storage paradigm. In an embodiment, genetic databases may contain raw sequence data, processed genetic markers, phenotypic associations, reference genomes, variant annotations, or other relevant information, and may be implemented using various software platforms and storage technologies.

Examples of database structures particularly suited for genetic data include: relational databases (such as MySQL or PostgreSQL) which organize genetic markers and their associations in tables with defined relationships; NoSQL databases (such as MongoDB) which provide flexible schemas for storing heterogeneous genetic data; graph databases (such as Neo4j) which can efficiently represent complex genetic relationships and inheritance patterns; columnar databases (such as Apache Cassandra) which efficiently store and query large volumes of sequence data; key-value stores (such as Redis) which enable rapid retrieval of specific genetic markers; and specialized genomic databases (such as the Genome Analysis Toolkit's GenomicsDB) which are optimized for variant data representation. The selection of database structure depends on factors including data volume, query patterns, analysis requirements, and performance considerations. In an embodiment, hybrid database architectures may also be employed, combining multiple paradigms to address the diverse requirements of genetic data management.

As used herein, the term “data structures” refers to specialized formats for organizing and storing data to enable efficient access and modification. In the context of genetic data, optimized data structures may include arrays, linked lists, trees (B-trees, binary trees), hash tables, graphs, tries, heaps, or other formats specifically designed for genetic sequence storage and retrieval. Efficient data structures are essential for managing large volumes of genetic data and enabling rapid search, comparison, and analysis operations.

In the context of the present disclosure, optimized data structures for storing and transferring genetic calls include standard bioinformatics formats. These comprise, but are not limited to, Variant Call Format (VCF) for representing single nucleotide polymorphisms, insertions, deletions, and structural variants; PLINK binary formats (e.g., .bed, .bim, .fam files) for high-performance extraction of allele dosages; and Hierarchical Data Format version 5 (HDF5) for the rapid, random-access querying of massive, multidimensional probabilistic genotype matrices.

As used herein, the term “processing unit” refers to any computational device or component capable of executing instructions, performing calculations, or processing data. In an embodiment, processing units may include central processing units (CPUs), graphics processing units (GPUs), field-programmable gate arrays (FPGAs), application-specific integrated circuits (ASICs), tensor processing units (TPUs), digital signal processors (DSPs), microcontrollers, or any other computational hardware, whether standalone, networked, or cloud-based.

As used herein, the term “heterosis” or “hybrid vigor” refers to the improved or increased function of any biological quality in a hybrid offspring, resulting from the mixing of genetic contributions of its parents. In an embodiment, heterosis may manifest as increased biomass, growth rate, fertility, yield, disease resistance, or other advantageous traits. The phenomenon occurs when the hybrid offspring displays qualities superior to those of either parent, particularly when the parents are genetically diverse but compatible.

As used herein, the term “trait optimization” refers to the process of selecting or modifying genetic configurations to maximize the expression of desirable traits while minimizing the expression of undesirable traits. In an embodiment, trait optimization may involve identifying genetic markers associated with specific traits, assessing the relative importance of different traits, predicting the outcome of genetic combinations, and selecting the most favorable combinations based on predetermined criteria. In an embodiment, trait optimization may be applied to physical characteristics, disease resistance, productivity, adaptability, or other heritable qualities.

As used herein, the term “genetic compatibility score” refers to a numerical value or set of values calculated to represent the predicted compatibility between two or more genetic profiles. The score is derived from weighted comparison of identified dominant and recessive traits, statistical modeling of potential genetic outcomes, assessment of genetic distance, and evaluation of complementary genetic markers. In an embodiment, a genetic compatibility score may indicate the likelihood of producing offspring with desired traits, the risk of heritable disorders, or other aspects of genetic compatibility.

As used herein, the term “optimal mating distance” or “OMD” refers to the ideal degree of genetic differentiation between reproductive partners that maximizes offspring fitness. The OMD balances the benefits of heterosis (hybrid vigor) from increased genetic diversity with the risks of genetic incompatibility from excessive genetic divergence. In an embodiment, the optimal mating distance may vary by species, population, or specific genetic context, and represents the genetic separation that produces the most favorable combination of heterosis and genetic compatibility.

As used herein, the term “F1 hybrid” refers to the first filial generation of offspring produced from a cross of two genetically distinct parental lines, breeds, or varieties. An F1 hybrid is a uniform composite receiving approximately 50% of its genome from each founder parent. In animal breeding, Fl hybrids are valued for their phenotypic uniformity and heterotic advantage relative to either parental line. In an embodiment, in plant breeding, F1 hybrids may exhibit enhanced yield, disease resistance, or environmental adaptability relative to either parental variety.

As used herein, the term “crossbreeding” refers to the deliberate mating of subjects from different breeds, strains, varieties, or genetically distinct lines. In an embodiment, crossbreeding may be performed in livestock, companion animals, or plants to capture heterosis for characteristics including, but not limited to, production, fertility, longevity, disease resistance, and environmental adaptability. The system disclosed herein facilitates optimized crossbreeding by computationally evaluating the predicted genetic compatibility, complementarity, and heterotic potential of candidate crosses prior to physical mating.

As used herein, the term “F1-selected crossbreed cross” refers to a cross between F0 subjects of different breeds wherein one or both of the F0 male and female gametes are from an F0 subject selected based on the performance of prior F1 progeny resulting from a cross of gametes from that F0 subject. In an embodiment, the system disclosed herein generates recommendations for F1-selected crossbreed crosses by evaluating F0 candidate pairings based on historical F1 progeny performance data, including but not limited to milk production, age at first calving, body depth, cell counts, cow conception rate, dairy form, daughter calving ease, daughter pregnancy rate, fat yield, feet and legs composite, final score, fore udder attachment, net merit, productive life, protein yield, rear udder height, rump angle, somatic cell score, stature, strength, udder composite, and udder depth. The concept of F1-selected crossbreed crosses, and systems and methods for generating and maintaining herds of hybrid dairy cattle through such crosses, are described in U.S. Pat. No. 10,159,226, which is incorporated herein by reference in its entirety.

As used herein, the term “two-line breeding system” refers to a breeding architecture comprising (1) a male line developed from elite breeds with genetic selection driven by performance of hybrid offspring, and (2) a female line developed from elite breeds with genetic selection driven by sustained female performance including oocyte yield and dairy production as well as performance of F1 offspring. In an embodiment, the system disclosed herein may implement or interface with a two-line breeding system, wherein the male line is maintained in a bull stud that produces semen, and the female line is maintained in an oocyte farm and female nucleus that provides both oocyte production for creation of F1 embryos and enables continuous genetic improvement of the female line. A testing system component allows high-quality performance information to be obtained from F1 animals in high-quality commercial dairy operations, providing selection data so that male and female line selections can be supported with F1 information and can diverge from breed-dependent selection. Such two-line breeding systems are described in U.S. Pat. No. 10,159,226, incorporated herein by reference.

As used herein, the term “multi-trait selection index” refers to a composite numerical value that integrates predicted transmitting abilities (PTAs), genomic estimated breeding values (GEBVs), or other genetic merit estimates for a plurality of traits, weighted by their relative economic or biological importance, to rank subjects for breeding purposes. Multi-trait selection indices applicable to dairy cattle include, but are not limited to, the Net Merit index (NM$), Cheese Merit index (CM$), Fluid Merit index (FM$), and Grazing Merit index (GM$). In an embodiment, the system may further incorporate Jersey Performance Index (JPI™) or analogous breed-specific composite indices. Multi-trait selection indices and their application to germplasm improvement are described in U.S. Pat. No. 10,975,351, which is incorporated herein by reference in its entirety. In an embodiment, the system disclosed herein distinguishes from conventional use of such indices by computing pair-specific predicted offspring distributions rather than relying on static, pre-computed population-level index values.

In an embodiment, the system may evaluate germplasm quality using one or more of the metrics and indices described in U.S. Pat. No. 10,975,351, including but not limited to predicted transmitting abilities for milk yield, fat yield, protein yield, productive life, somatic cell score, daughter pregnancy rate, cow conception rate, heifer conception rate, cow livability, calving ease, stillbirth rate, stature, dairy form, rump angle, rear legs side view, fore udder height, fore udder attachment, rear udder height, rear udder width, udder depth, front teat placement, teat length, and udder composite index. In an embodiment, the system further evaluates haplotype status for known deleterious recessive haplotypes, including but not limited to JH1 and JH2 in Jersey cattle, HH1 through HH6 in Holstein cattle, and analogous haplotypes identified in other breeds, as described in U.S. Pat. No. 10,975,351 and as set forth herein.

As used herein, the term “selection index” refers to a composite numerical value that combines multiple trait evaluations, weighted by their relative economic or biological importance, to rank subjects for breeding purposes. Examples of selection indices utilized in dairy cattle breeding include, but are not limited to, the Net Merit index (NM$), which estimates lifetime profit based on incomes and expenses relevant for dairy producers; the Cheese Merit Index (CM$), which is optimized for milk sold for cheese production; the Fluid Merit Index (FM$), which is designed for markets where protein is not directly compensated; and the Grazing Merit Index (GM$), which is geared toward herds on pasture systems. In an embodiment, traits incorporated into such indices may comprise protein yield, fat yield, productive life (PL), somatic cell score (SCS), udder composite, feet/legs composite, body size composite, daughter pregnancy rate (DPR), cow conception rate (CCR), heifer conception rate (HCR), cow livability (LIV), calving ability, and other economically relevant traits. In an embodiment, in human fertility applications, analogous composite indices may incorporate polygenic risk scores, monogenic disease carrier burden, immune compatibility metrics, and other clinically relevant parameters. In an embodiment, the system disclosed herein may utilize such multi-trait selection indices as inputs to, or outputs of, the genetic compatibility assessment engine.

As used herein, the term “predicted transmitting ability” or “PTA” refers to the estimated genetic merit of a subject expressed as the predicted difference in performance of its progeny from the population average. PTAs are commonly computed for traits including milk yield, fat yield, protein yield, productive life, somatic cell score, daughter pregnancy rate, and various linear type traits. In an embodiment, PTAs may be derived from pedigree-based evaluation, genomic evaluation, or a combination thereof.

As used herein, the term “genomic estimated breeding value” or “GEBV” refers to the breeding value of a subject estimated using genome-wide marker information, typically from single nucleotide polymorphism (SNP) arrays or whole-genome sequencing data. GEBVs enable the prediction of genetic merit without requiring progeny testing, thereby reducing the generation interval and accelerating genetic gain in breeding programs.

As used herein, the term “recessive haplotype” refers to a chromosomal segment carrying one or more alleles that, when present in a homozygous state, may result in reduced fertility, embryonic lethality, or phenotypic defects. In dairy cattle, known deleterious recessive haplotypes include, but are not limited to, JH1 and JH2 in Jersey cattle, HH1 through HH6 in Holstein cattle, and analogous haplotypes identified in other breeds. The system disclosed herein identifies carrier status for such recessive haplotypes and computes the probability of producing homozygous offspring for any given candidate pairing, thereby enabling the avoidance or minimization of matings that carry elevated risk of embryonic loss or neonatal defects.

As used herein, the term “recessive risk screening” refers to the process of identifying and evaluating genetic variants that may cause disease or undesirable traits when present in homozygous form (two copies). Recessive risk screening involves analyzing genetic profiles to detect carrier status for recessive conditions, assessing the probability of transmission to offspring, and evaluating compatibility between potential reproductive partners to minimize the risk of recessive trait expression in offspring.

As used herein, the term “bioinformatics database” refers to a specialized database designed for storing, organizing, and analyzing biological and genetic data. In an embodiment, bioinformatics databases may contain genomic sequences, protein structures, metabolic pathways, gene expression data, phenotypic associations, or other biological information. In an embodiment, these databases typically include specialized data structures, indexing methods, and query capabilities optimized for biological data types and may be integrated with analytical tools for data processing and interpretation.

As used herein, the term “immutable distributed ledger” refers to a decentralized, cryptographically secured database (such as a blockchain) maintained across a network of nodes, wherein records cannot be altered retroactively without the alteration of all subsequent blocks.

As used herein, “cryptographic hashing” refers to the process of mapping genetic sequence data, compatibility scores, and matching recommendations through a mathematical algorithm (e.g., SHA-256) to output a fixed-size alphanumeric string. Recording these hashes on the distributed ledger establishes a mathematically verifiable, tamper-evident chain of provenance for elite genetic lines, ensuring the integrity of the data inputs utilized by the system's predictive algorithms.

As used herein, the term “Next-Generation Sequencing” or “NGS” refers to high-throughput DNA sequencing technologies that can sequence multiple DNA fragments in parallel, producing thousands or millions of sequences concurrently. NGS methods include, but are not limited to, sequencing by synthesis, ion semiconductor sequencing, pyrosequencing, sequencing by ligation, and nanopore sequencing. NGS enables more comprehensive genetic analysis than traditional Sanger sequencing and serves as a primary data acquisition method for genetic profiling. Performing NGS creates sequencing data.

Sequencing data may be stored in any suitable format. In one embodiment, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting variants called from the sequence data and release it for reporting to subjects, health care providers, and/or scientists.

As used herein, the term “Single Nucleotide Polymorphisms” or “SNPs” refers to variations at single positions in a DNA sequence among subjects. SNPs represent a difference in a single nucleotide—A, T, C, or G—at a specific position in the genome. In an embodiment, these variations may occur in coding regions (exons), non-coding regions (introns, regulatory regions), or intergenic regions, and may influence protein structure, gene expression, or other biological functions. SNPs are the most common type of genetic variation and serve as important genetic markers.

As used herein, the term “Short Tandem Repeats” or “STRs” refers to microsatellite DNA sequences consisting of repeating units of 2-6 base pairs. The number of repeat units at specific loci varies among subjects, making STRs useful as genetic markers for identification, paternity testing, and population genetics. In an embodiment, STRs may occur in coding or non-coding regions and can influence gene expression, protein function, or disease risk when the number of repeats exceeds normal ranges.

As used herein, the term “epigenetic markers” refers to heritable modifications to DNA or associated proteins that affect gene expression without altering the underlying DNA sequence. Epigenetic markers include, but are not limited to, DNA methylation, histone modifications, chromatin remodeling, and non-coding RNA interactions. In an embodiment, these markers influence how genes are expressed during development and in response to environmental factors, and they may be inherited through cell division or, in some cases, transmitted to offspring.

As used herein, the term “predefined genetic inheritance models” refers to established frameworks that describe how genetic traits are transmitted from one generation to the next. These models include, but are not limited to, Mendelian inheritance patterns (dominant, recessive, co-dominant, incomplete dominance), polygenic inheritance, multifactorial inheritance, maternal inheritance, genomic imprinting, and other mechanisms of genetic transmission. Predefined genetic inheritance models provide the theoretical basis for predicting the inheritance of specific traits based on genetic profiles.

As used herein, the term “weighted comparison” refers to an analytical method that assigns different levels of importance or influence on various factors during comparison or evaluation. In an embodiment, in genetic analysis, weighted comparison may involve assigning higher importance to certain genetic markers based on their known impact on traits of interest, reliability of association, effect size, or relevance to specific objectives. Weighted comparison allows for more nuanced assessment of genetic compatibility by reflecting the relative significance of different genetic features.

As used herein, the term “statistical probability models” refers to mathematical frameworks used to calculate the likelihood of specific genetic outcomes based on identified genetic markers and their known inheritance patterns. In an embodiment, these models may incorporate Bayesian statistics, logistic regression, Monte Carlo simulations, Markov models, or other probabilistic approaches to predict the likelihood of trait expression, disease risk, or other genetic outcomes. Statistical probability models account for the inherent uncertainty in genetic inheritance and enable risk assessment and outcome prediction.

As used herein, the term “Bayesian inference” refers to a method of statistical inference in which Bayes' theorem is used to update the probability for a hypothesis as more evidence or genetic information becomes available. In the context of genetic analysis, Bayesian inference is utilized to estimate the posterior distributions of additive genetic effects, dominance effects, and penetrance parameters by combining prior knowledge (e.g., historical trait distributions) with newly acquired genomic sequencing data. In an embodiment, the system relies on Bayesian statistical modeling to estimate parameters for traits and predict outcomes. Bayesian inference utilizes Bayes' theorem to update the probability for a hypothesis as more evidence or information becomes available. In an aspect, posterior distributions of marker effects, trait parameters, and breeding values are estimated using Bayesian inference methods, which may include, but are not limited to, Markov chain Monte Carlo (MCMC) algorithms (such as Gibbs sampling or Metropolis-Hastings), variational inference, or approximate Bayesian computation (ABC).

As used herein, the term “Monte Carlo simulation” refers to a broad class of computational algorithms that rely on repeated random sampling to obtain numerical results. In the present system, Monte Carlo simulations are utilized to propagate parental genotype uncertainty and posterior parameter uncertainty into predicted offspring outcome distributions by iteratively sampling genotypes and trait effects over a plurality of iterations (e.g., 10,000 to 100,000 iterations). In an embodiment, the system utilizes simulation techniques to propagate genotype uncertainty and Bayesian parameter uncertainty into predicted offspring outcome distributions. In an aspect, this is achieved using Monte Carlo simulation, which relies on repeated random sampling to obtain numerical results. While Monte Carlo simulation is a preferred method, alternative stochastic modeling and uncertainty propagation techniques may be employed, including Latin Hypercube sampling, bootstrapping methods, deterministic analytical approximations (such as Taylor series expansion or unscented transforms), or scenario-based tree analysis.

As used herein, the term “combinatorial optimization” refers to the process of searching for an optimal object from a finite set of objects. In the context of cohort-level mating assignments, it involves utilizing mathematical algorithms (such as integer programming, mixed-integer linear programming, or genetic algorithms) to select a portfolio of genetic crosses that maximizes a defined multi-objective function while simultaneously satisfying discrete operational constraints (e.g., maximum inseminations per sire, or minimum herd diversity thresholds).

As used herein, the term “sequence alignment algorithm” refers to a computational method used to align and compare genetic sequences to identify similarities, differences, and genetic markers. In an embodiment, sequence alignment algorithms may include global alignment algorithms (e.g., Needleman-Wunsch), local alignment algorithms (e.g., Smith-Waterman), heuristic algorithms (e.g., BLAST, FASTA), multiple sequence alignment algorithms (e.g., ClustalW, MUSCLE), and specialized algorithms for next-generation sequencing data. These algorithms enable the identification of shared sequences, variations, insertions, deletions, and other features across genetic sequences.

As used herein, the term “dominant trait” refers to a genetic trait that is expressed when at least one allele associated with the trait is present in a subject's genetic profile. Dominant traits are manifested in the phenotype when inherited from just one parent, and the presence of a dominant allele can mask the expression of a recessive allele at the same locus.

As used herein, the term “recessive trait” refers to a genetic trait that is expressed only when two copies of the allele associated with the trait are present in a subject's genetic profile (homozygous recessive). Recessive traits are manifested in the phenotype only when inherited from both parents and are not expressed in the presence of a dominant allele.

As used herein, the term “sequence matching matrix” refers to a two-dimensional array used in sequence alignment algorithms to compute similarity scores between nucleotide or amino acid sequences. Each cell in the matrix represents the score for aligning specific positions in the sequences being compared, and the matrix is populated according to scoring criteria that reward matches and penalize mismatches or gaps.

As used herein, the term “genetic distance” refers to the degree of genetic differentiation or divergence between subjects, populations, or species, typically measured by the number or proportion of genetic differences. In an embodiment, genetic distance may be calculated based on nucleotide differences, allele frequencies, or other genetic markers, and serves as an indicator of evolutionary relatedness or genetic compatibility.

As used herein, the term “genetic diversity” refers to the variety of genetic characteristics within a species, population, or gene pool. Genetic diversity encompasses all forms of genetic variation, including allelic diversity, structural variation, and epigenetic differences, and serves as a critical factor in population resilience, adaptation, and evolutionary potential.

As used herein, the term “genome-wide association study” or “GWAS” refers to an approach that involves rapidly scanning markers across complete sets of DNA, or genomes, of many people to find genetic variations associated with a particular disease or trait. These studies typically focus on associations between single-nucleotide polymorphisms (SNPs) and traits like major human diseases or phenotypic characteristics.

As used herein, the term “machine learning model” refers to a computational system that improves its performance on a specific task through experience or data. In an embodiment, machine learning models used in genetic analysis may include supervised learning algorithms (e.g., support vector machines, random forests), unsupervised learning algorithms (e.g., clustering methods), deep learning networks, reinforcement learning systems, or hybrid approaches.

As used herein, the term “genetic algorithm” refers to a search heuristic inspired by natural selection that can be used to find approximate solutions to optimization and search problems. Genetic algorithms evolve solutions through mechanisms inspired by biological evolution, including selection, crossover, and mutation, and can be applied to optimize genetic selection or breeding strategies.

As used herein, the term “genomic selection” refers to a form of marker-assisted selection that uses genetic markers covering the entire genome to predict the breeding value of a subject. Genomic selection employs statistical methods to estimate the effects of all genetic markers simultaneously, without necessarily identifying the specific genes contributing to a trait.

As used herein, the term “breeding value” refers to the genetic merit of a subject as a parent, representing the expected performance of its offspring relative to the population average. Breeding value is determined by the additive genetic effects that a subject can transmit to its offspring and serves as a key metric in selective breeding programs.

As used herein, the term “genetic gain” refers to the improvement in the average genetic merit of a population over generations as a result of selection. Genetic gain is influenced by selection intensity, genetic variance, selection accuracy, and generation interval, and represents the rate of progress in breeding programs.

As used herein, the term “genetic marker index” refers to a data structure or reference system that organizes genetic markers for efficient storage, retrieval, and analysis. In an embodiment, a genetic marker index may be implemented using various data structures and algorithms optimized for the specific characteristics and usage patterns of genetic data.

The terms “about” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “including” is understood to mean “including but not limited to.” “Including” and “including but not limited to” are used interchangeably.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

In an embodiment, reproductive technologies are integrated into the optimization platform. “In Vitro Fertilization” (IVF) refers to a method of fertilizing an egg outside of a living organism, typically involving the aspiration of oocytes from a donor, maturation, and co-culture with selected spermatozoa. “Artificial Insemination” (AI) refers to the introduction of semen into a female's reproductive tract by hand to achieve pregnancy without natural service. In an aspect, the semen utilized may be sex-selected sperm, which has been separated into subpopulations containing X-chromosome bearing sperm and Y-chromosome bearing sperm having a purity in the range of about 70 percent to about 100 percent. In a further embodiment, the platform generates recommendations for “Somatic Cell Nuclear Transfer” (SCNT), a process wherein the nucleus of a somatic cell is transferred into an enucleated oocyte to propagate genetically superior animals.

In an embodiment, the system accounts for and manages genetic modifications, including “gene-edited” organisms. In an aspect, gene editing is accomplished utilizing targeted nucleases, including, but not limited to, Clustered Regularly Interspaced Short Palindromic Repeats (CRISPR)/Cas9 systems, Transcription Activator-Like Effector Nucleases (TALENs), Zinc Finger Nucleases (ZFNs), a recombinase fusion protein, or a meganuclease to introduce precise chromosomal sequence modifications.

In an embodiment, the genetic compatibility assessment engine utilizes multi-trait selection indices to evaluate candidate pairings. In an aspect relating to dairy cattle, such indices comprise a Net Merit index (NM$), which estimates lifetime profit based on incomes and expenses relevant for dairy producers. In an embodiment, traits included in NM$ calculations may comprise protein yield, fat yield, productive life (PL), somatic cell score (SCS), udder composite, feet/legs composite, body size composite, and daughter pregnancy rate (DPR). In an embodiment, other indices may include a Cheese Merit Index (CM$) optimized for milk sold to be made into cheese, a Fluid Merit Index (FM$) for markets where protein is not directly compensated, or a Grazing Merit Index (GM$) geared toward herds on pasture systems demanding higher fertility.

As used herein, the term “complementary index” refers to a multi-objective scoring metric that combines multiple distribution-derived measurements into a single comparative value for evaluating candidate genetic crosses or pairings. In an aspect, the complementary index mathematically blends one or more of: expected economic values (e.g., predicted yield, carcass index, or net merit); expected phenotypic performance values derived from posterior predictive distributions; penalty terms for identified risks (e.g., dystocia risk, stillbirth risk, probability of deleterious homozygosity, or elevated genomic relatedness); dominance or heterozygosity benefit terms that quantify the predicted hybrid vigor resulting from the cross; immune compatibility metrics (e.g., HLA complementarity in humans or BoLA diversity in cattle); and tail-risk metrics derived from the lower quantiles of the offspring trait distributions. The complementary index serves as an objective function for ranking subject pairings or as an input to cohort-level combinatorial optimization.

In an embodiment, candidate crosses and pairings are evaluated using a “complementary index.” A complementary index is a multi-objective scoring metric that combines multiple distribution-derived measurements into a single comparative value. In an aspect, the complementary index mathematically blends expected economic values (e.g., predicted yield or carcass index) with penalty terms for identified risks (e.g., dystocia risk, stillbirth risk, probability of deleterious homozygosity, or high genomic relatedness). In an embodiment, the complementary index may further comprise a dominance or heterozygosity benefit term to quantify the predicted hybrid vigor resulting from the cross.

In an embodiment, the platform generates cohort-level assignments and portfolios of crosses. To solve these complex assignments under operational constraints, the system may employ combinatorial optimization techniques. In an aspect, these techniques comprise integer programming, mixed-integer programming (MIP), linear programming, or metaheuristic algorithms such as evolutionary algorithms, genetic algorithms, simulated annealing, or particle swarm optimization.

As used herein, the term “uncertainty propagation” refers to the computational process of determining the uncertainty in outputs of a system based on the uncertainties in its inputs. In the context of genetic compatibility assessment, uncertainty propagation encompasses methods for translating genotype uncertainty (e.g., from imputed or low-coverage sequencing data) and parameter estimation uncertainty (e.g., from Bayesian posterior distributions of marker effects) into predicted outcome distributions for offspring traits. In an embodiment, the system may employ one or more uncertainty propagation methods including, but not limited to: Monte Carlo simulation, in which repeated random sampling from input distributions generates an empirical output distribution; Latin Hypercube sampling, a stratified sampling variant that provides more efficient coverage of the input space; quasi-Monte Carlo methods utilizing low-discrepancy sequences (e.g., Sobol or Halton sequences) for improved convergence rates; bootstrapping methods, in which resampled datasets are used to estimate the sampling distribution of a statistic; deterministic analytical approximations such as first-order or second-order Taylor series expansion (delta method), which propagate variance through linearized model equations; unscented transforms, which use a deterministic set of sigma points to capture the mean and covariance of the output distribution; polynomial chaos expansion, which creates a surrogate model using polynomials to map input uncertainty to output uncertainty, and scenario-based tree analysis, in which a discrete set of representative scenarios spans the range of plausible outcomes.

As used herein, the term “Markov chain Monte Carlo” or “MCMC” refers to a class of algorithms for sampling from probability distributions that are difficult to sample from directly. MCMC methods construct a Markov chain whose stationary distribution is the target posterior distribution, enabling estimation of posterior means, variances, quantiles, and credible intervals for model parameters. In an embodiment, MCMC algorithms employed by the system may include, but are not limited to, Gibbs sampling, Metropolis-Hastings algorithms, Hamiltonian Monte Carlo (HMC), No-U-Turn Sampler (NUTS), and slice sampling. In an embodiment, convergence diagnostics including, but not limited to, Gelman-Rubin statistics, effective sample size, and trace plot analysis may be employed to assess chain convergence.

As used herein, the term “variational inference” refers to a family of approximate Bayesian inference methods that cast the problem of computing posterior distributions as an optimization problem. Variational inference methods approximate the true posterior distribution with a simpler, parameterized family of distributions by minimizing the Kullback-Leibler divergence between the approximate and true posterior. In an embodiment, variational inference may provide computational advantages over MCMC sampling for large-scale genomic datasets while providing reasonable approximations to the full posterior distribution.

As used herein, the term “approximate Bayesian computation” or “ABC” refers to a class of computational methods that enable Bayesian inference when the likelihood function is intractable or computationally expensive to evaluate. ABC methods simulate data from the model using proposed parameter values and compare simulated summary statistics to observed summary statistics to approximate the posterior distribution. In an embodiment, ABC methods may be employed by the system when the genetic models underlying trait prediction are sufficiently complex that exact likelihood computation is infeasible.

As used herein, the term “multi-objective optimization” refers to an optimization approach that simultaneously considers multiple, potentially conflicting objectives. In the context of breeding program management, multi-objective optimization may balance competing goals such as maximizing short-term genetic gain, maintaining long-term genetic diversity, minimizing inbreeding accumulation, and satisfying operational constraints. In an embodiment, multi-objective optimization methods employed by the system may include, but are not limited to, weighted sum approaches, Pareto-optimal frontier methods, epsilon-constraint methods, and evolutionary multi-objective algorithms such as NSGA-II (Non-dominated Sorting Genetic Algorithm II) or MOEA/D (Multi-Objective Evolutionary Algorithm based on Decomposition).

### System Architecture Overview

Field-Programmable Gate Arrays, commonly known as FPGAs, are a powerful hardware platform for accelerating computationally intensive data analysis tasks. Unlike general-purpose processors, FPGAs consist of an array of configurable logic blocks and programmable interconnects that can be tailored to execute specific algorithms with exceptional efficiency. This reconfigurability allows researchers to design custom hardware pipelines that process data in a massively parallel fashion, achieving significant speedups over traditional CPU-based approaches. As the volume and complexity of data continue to grow across scientific disciplines, FPGAs offer a compelling balance of performance, power efficiency, and flexibility that makes them well-suited for demanding analytical workloads.

One of the most promising applications of FPGA technology lies in the field of genetic data analysis. Modern genomic research generates enormous datasets, particularly through next-generation sequencing technologies that produce billions of short DNA reads per run. Aligning these reads to a reference genome, identifying variants, and performing downstream statistical analyses are tasks that require substantial computational resources. FPGAs can be programmed to implement the core algorithms used in these processes, such as the Burrows-Wheeler Transform for sequence alignment or hidden Markov models for variant calling, directly in hardware. By doing so, these chips can process genomic data orders of magnitude faster than software running on conventional processors, dramatically reducing the time required to move from raw sequencing output to actionable biological insights.

The architectural advantages of FPGAs are particularly well-matched to the characteristics of genetic data processing. Genomic analysis pipelines often involve repetitive operations applied uniformly across massive datasets, a pattern that lends itself naturally to the fine-grained parallelism that FPGAs provide. Unlike graphics processing units, which also offer parallel computation, FPGAs allow developers to customize the data path and memory access patterns at the hardware level, minimizing bottlenecks and maximizing throughput for specific bioinformatics algorithms. Additionally, FPGAs consume significantly less power than GPU clusters of comparable performance, an important consideration for large-scale sequencing centers and cloud-based genomics platforms that must manage both computational throughput and energy costs.

Despite their advantages, the adoption of FPGAs for genetic data analysis is not without challenges. Programming FPGAs traditionally requires expertise in hardware description languages such as VHDL or Verilog, which presents a steep learning curve for bioinformaticians and data scientists accustomed to high-level programming environments. However, this barrier is steadily diminishing as high-level synthesis tools and open-source genomics acceleration frameworks continue to mature, enabling researchers to deploy FPGA-accelerated pipelines without deep hardware engineering knowledge. As sequencing costs decline and the demand for rapid, large-scale genomic analysis intensifies in areas such as precision medicine, population genomics, and clinical diagnostics, FPGAs are poised to play an increasingly central role in the computational infrastructure that underpins modern genetic research.

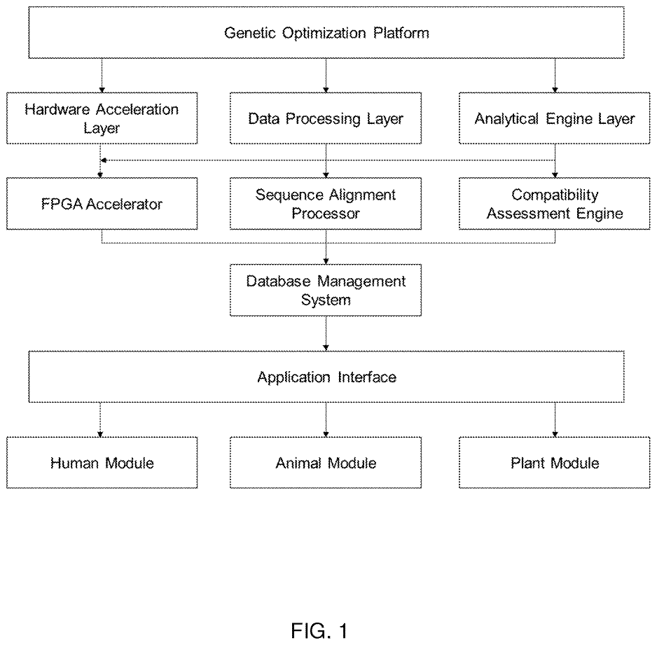

The system for genetic profile analysis and compatibility assessment employs a multi-layered architecture designed to efficiently process genetic data across diverse applications. A specialized hardware infrastructure lies at the foundation of this architecture (FIG. 1), leveraging field-programmable gate arrays (FPGAs) to accelerate computation-intensive genetic sequence operations. This hardware layer interfaces with a comprehensive software stack that implements the analytical and decision-making functions of the system.

The architectural framework comprises four primary layers: (1) a hardware acceleration layer, (2) a data processing layer, (3) an analytical engine layer, and (4) an application interface layer. The hardware acceleration layer contains the FPGAs configured to implement sequence alignment algorithms and other computationally demanding genetic analysis operations. The data processing layer manages data acquisition, quality control, and preliminary processing of genetic sequence data. The analytical engine layer houses the core algorithms for genetic profile analysis, trait classification, compatibility assessment, and risk evaluation. The application interface layer provides domain-specific implementations for human, animal, and plant applications.

Data flows through this architecture in a defined processing pipeline, beginning with raw genetic data acquisition and culminating in actionable compatibility assessments or trait optimization recommendations. The system employs a modular design that allows components to be upgraded or replaced independently, facilitating technological advancement without requiring complete system redesign. Each module communicates through standardized interfaces, enabling efficient data transfer while maintaining functional independence.

In an embodiment, rather than prescriptively defining optimal genetic outcomes, the system may focus on identifying and mitigating potential genetic risks, providing a framework that eliminates identifiable problems while preserving the natural role of chance in genetic recombination and development. This approach acknowledges the complexity of genetic inheritance and avoids oversimplified determinations of what constitutes “best” genetic profiles.

### Processing Pipeline Architecture

The system implements a multi-stage processing pipeline architecture that enables efficient processing of genetic data from raw sequence information to compatibility assessment results. The pipeline architecture includes dedicated stages for sequence preprocessing, marker identification, trait classification, compatibility scoring, and risk assessment. Each stage processes data and passes results to subsequent stages with minimal latency, enabling high throughput operation.

The pipeline architecture includes feedback paths that allow later stages to request additional processing from earlier stages when anomalous genetic patterns are detected. This adaptive processing approach ensures comprehensive analysis of complex genetic interactions without requiring excessive computational resources for routine cases. The pipeline architecture incorporates checkpoint mechanisms that periodically save intermediate results, enabling recovery from processing failures without restarting the entire analysis.

In some embodiments, the pipeline architecture implements flow control mechanisms that regulate data movement between stages to prevent bottlenecks and ensure optimal resource utilization. These mechanisms include buffer management systems that dynamically adjust buffer sizes based on observed processing patterns and data characteristics.

### Hardware Implementation

### FPGA Configuration for Genetic Analysis

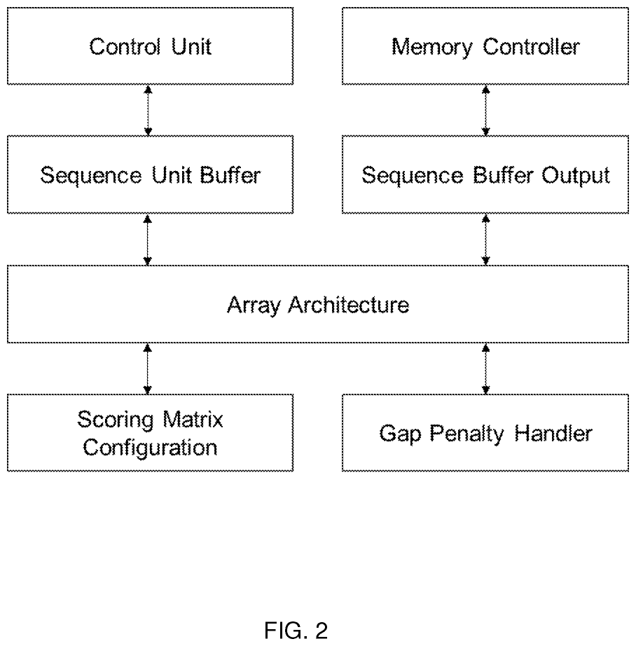

The system employs Field-Programmable Gate Arrays (FPGAs) specifically configured to accelerate genetic sequence alignment algorithms (FIG. 2). These FPGAs are programmed with custom bitstreams that implement hardware-optimized versions of sequence analysis algorithms such as Smith-Waterman or Needleman-Wunsch. The FPGA configuration includes dedicated logic blocks for parallel computation of nucleotide scoring matrices, which substantially increases the throughput of genetic sequence comparisons.

In an embodiment, the FPGA implements an array architecture specifically designed for sequence alignment (e.g., a systolic array), wherein each processing element in the array performs nucleotide comparison operations in parallel. This architecture is particularly well-suited for the dynamic programming approach used in sequence alignment algorithms, as it maps the computational dependencies directly onto the hardware structure. The FPGA configuration includes specialized modules for handling different genetic marker types, including Single Nucleotide Polymorphisms (SNPs), Copy Number Variations (CNVs), and Short Tandem Repeats (STRs). These modules are interconnected through a customized datapath that minimizes data transfer overhead between processing stages.

The FPGA configuration incorporates hardened error detection and correction circuits to ensure the accuracy of genetic analysis results. These circuits implement cyclic redundancy check (CRC) algorithms or more advanced error correction codes specifically tailored for genetic data processing. By implementing these error-correction mechanisms in hardware, the system maintains data integrity without imposing additional computational overhead on the host system.

### Parallel Processing Architecture

The system implements a hierarchical parallel processing architecture that enables simultaneous analysis of multiple genetic sequences. The architecture includes multiple processing layers, each optimized for specific genetic analysis operations. The first layer implements coarse-grained parallelism, where different genetic samples are processed simultaneously by separate processing units. The second layer implements fine-grained parallelism, where subject genetic markers within a sample are analyzed in parallel.

The parallel processing architecture includes a workload distribution subsystem that dynamically allocates computational resources based on the complexity of the genetic analysis tasks. This subsystem employs scheduling algorithms that prioritize time-critical operations such as dominant trait identification and recessive risk screening, ensuring optimal system throughput while maintaining analysis accuracy.

In some embodiments, the parallel processing architecture incorporates specialized processing elements arranged in a pipeline configuration, where each element is optimized for a specific genetic analysis operation. These operations include sequence alignment, variant calling, trait classification, and compatibility scoring. The processing elements communicate through dedicated high-speed interconnects that minimize data transfer latency.

### Memory Subsystems Design

The system includes hierarchical memory subsystems specifically designed to efficiently store and retrieve genetic data. These subsystems include high-bandwidth, low-latency memory modules positioned close to the processing elements to store frequently accessed genetic markers and reference sequences. The memory subsystem employs specialized data structures such as bloom filters, hash tables, or B-trees optimized for rapid genetic marker lookup.

The memory subsystems implement hybrid memory architectures that combine different memory technologies based on access patterns and data persistence requirements. For example, volatile memory (such as DRAM) is used for temporary storage of intermediate results, while non-volatile memory (such as NAND flash) is used for persistent storage of genetic profiles and trait databases. This hybrid approach optimizes both performance and data persistence while accommodating the diverse access patterns characteristic of genetic analysis workloads.

In some embodiments, the memory subsystems incorporate dedicated hardware for data compression and decompression, reducing the memory footprint of genetic data without sacrificing access speed. These hardware accelerators implement genetic-specific compression algorithms that exploit the inherent redundancy in genomic sequences, achieving higher compression ratios than general-purpose compression techniques. The compression hardware operates transparently to the processing elements, automatically compressing data when writing to memory and decompressing when reading, thereby maintaining computational efficiency while reducing memory bandwidth requirements.

### Genetic Profile Analysis Methods

### DNA Matching and Complementary Profile Identification

The system's technical approach to DNA matching goes beyond simple sequence alignment to identify complementary genetic profiles based on dominant and recessive trait analysis. This involves specialized algorithms that evaluate how well two genetic profiles function together in offspring, not merely identifying genetic similarity.

The DNA matching process begins with comprehensive genetic profiling of potential donors and recipients. The system extracts genetic data from gametes (sperm or ova) or tissue samples using next-generation sequencing methods. The hardware-accelerated sequence alignment processor aligns this data to reference genomes with significantly improved processing speed compared to software-only implementations.

For human and animal applications, the system implements algorithms to:
Identify markers associated with dominant traits such as eye color, hair color, disease resistance, and metabolic efficiency;
Screen for recessive traits linked to hereditary risks, ensuring complementary pairing to minimize risk; and
Evaluate compatibility at critical genetic loci, such as HLA (Human Leukocyte Antigen) genes in humans or immune-response markers in animals.

For plant applications, the system analyzes genetic markers to:
Identify traits related to disease resistance, yield, and environmental adaptability;
Select complementary genetic profiles to maximize hybrid vigor (heterosis); and
Optimize cross-pollination strategies based on genetic compatibility.

The DNA matching algorithms implement a weighted scoring system that balances multiple factors:
Genetic distance calculation to measure overall genetic similarity;
Optimal mating distance determination to balance diversity and compatibility;
Recessive risk evaluation to minimize hereditary disease transmission; and
Dominant trait prioritization to enhance desired characteristics.

The system's DNA matching capability extends beyond gamete analysis to comprehensive matching of genetic profiles from any DNA source, enabling applications such as mate selection, family relationship verification, and genetic compatibility assessment between subjects at any life stage. This broader DNA matching functionality analyzes complete genetic profiles rather than just reproductive cells, identifying complementary genetic patterns that would function optimally together when combined.

For genetic compatibility assessment applications, the system implements specialized algorithms that evaluate genetic interactions between potential partners based on their complete DNA profiles. These algorithms assess the probability of various genetic outcomes in potential offspring, identify hereditary disease risks, and evaluate the complex interplay of genetic factors that influence offspring development, acknowledging that the process involves both genetic assessment and the inherent role of chance in genetic recombination. In an embodiment, the genetic compatibility process incorporates analysis of genetic carrier status for recessive disorders, identifying situations where both partners carry recessive disease alleles. It also evaluates immune system compatibility, particularly at Human Leukocyte Antigen (HLA) loci, which may influence reproductive success and offspring health. Additionally, the system examines polygenic trait combinations that enhances offspring fitness or predispose to complex conditions, along with genetic diversity metrics that predict the benefits of heterosis (hybrid vigor) in offspring.

The DNA matching system calculates optimal genetic distance between potential mates, finding the ideal balance point where genetic diversity provides maximum benefit while avoiding incompatibility issues that can occur when genetic backgrounds are too divergent. This optimal mating distance calculation is implemented through statistical models that analyze the distribution of genetic variations across the genome and predict their combined effects.

In an embodiment, the optimal mating distance (OMD) is calculated by first determining the genomic relationship matrix (G-matrix) or Identity-by-State (IBS) distance between the potential mates. The genetic distance (D) is calculated utilizing allele frequencies across the evaluated loci. The system then maps this genetic distance to a quadratic or non-linear fitness function, F(D)=aD−bD²+C, where a represents the linear benefit of heterosis and b represents the exponential penalty of outbreeding depression or genetic incompatibility. The OMD is identified mathematically as the vertex of this fitness function, representing the precise genetic distance that maximizes the predicted offspring fitness score before incompatibility penalties degrade viability.

This multi-factor analysis is implemented through specialized hardware-accelerated algorithms that can process thousands of genetic markers simultaneously, achieving computational efficiency not possible with general-purpose computing architectures.

The DNA matching system addresses the challenge of genetic preservation without inbreeding, particularly relevant for conservation genetics and small populations. In an embodiment, the optimal mating distance calculation may incorporate inbreeding coefficients and relatedness metrics to prevent excessive genetic similarity while preserving valuable genetic traits. In an embodiment, for conservation applications, the system may identify mating pairs that maintain genetic integrity of endangered populations while sufficiently increasing genetic diversity to avoid inbreeding depression. In an embodiment, this balance is achieved through algorithms that may evaluate both overall genetic distance and specific functional genetic regions, ensuring that mates preserve essential adaptive traits while introducing sufficient genetic diversity to maintain population health.

### Sequence Alignment Algorithms

The system incorporates a hardware-optimized implementation of the Smith-Waterman algorithm for local sequence alignment. This implementation utilizes specialized circuitry in FPGAs that realize the dynamic programming matrix calculations with significantly higher efficiency than general-purpose processors. The Smith-Waterman implementation includes affine gap penalty handling, which provides more biologically relevant alignment results by differentiating between gap openings and extensions.

The implementation supports configurable scoring matrices that can be tailored to specific alignment scenarios, such as DNA-to-DNA alignment, protein sequence alignment, or cross-species comparison. The Smith-Waterman implementation incorporates heuristic pre-filtering stages that identify promising alignment regions, enabling the computationally intensive precise alignment to focus on the most relevant portions of the sequences. This approach substantially increases throughput while maintaining alignment accuracy.

The implementation includes specialized memory access patterns that optimize cache utilization and minimize memory bandwidth requirements, further enhancing performance. The system implements massively parallel architectures for computing sequence matching matrices, a fundamental operation in genetic sequence alignment. These architectures distribute matrix computation across multiple processing elements that operate concurrently, dramatically reducing the time required for alignment operations.

In an embodiment directed to human fertility applications, the system evaluates genetic markers obtainable from one or more publicly available databases. In an aspect, the system evaluates markers at a minimum of 1,000 loci, with the evaluated loci organized by functional category, including but not limited to:
(a) Monogenic recessive disorder loci: variants cataloged in ClinVar (accessible at ncbi.nlm.nih.gov/clinvar) and the Online Mendelian Inheritance in Man (OMIM) database (accessible at omim.org) associated with over 6,000 known monogenic disorders, including but not limited to cystic fibrosis (CFTR gene), sickle cell disease (HBB gene), Tay-Sachs disease (HEXA gene), spinal muscular atrophy (SMN1 gene), and phenylketonuria (PAH gene);
(b) Polygenic risk score loci: variants identified through genome-wide association studies (GWAS) cataloged in the NHGRI-EBI GWAS Catalog (accessible at ebi.ac.uk/gwas) and associated with complex diseases including cardiovascular disease, type 2 diabetes, neurodegenerative conditions, and psychiatric disorders;
(c) Immune compatibility loci: Human Leukocyte Antigen (HLA) loci including HLA-A, HLA-B, HLA-C, HLA-DRB1, HLA-DQB1, and HLA-DPB1, and killer-cell immunoglobulin-like receptor (KIR) gene variants;
(d) Pharmacogenomic loci: variants cataloged in the Pharmacogenomics Knowledge Base (PharmGKB) that influence drug metabolism and therapeutic response; and
(e) Population-level allele frequency data: allele frequencies across diverse human populations as cataloged in the Genome Aggregation Database (gnomAD, accessible at gnomad.broadinstitute.org).

In an embodiment directed to crop and agricultural plant applications, the system evaluates genetic markers at a plurality of loci, with the evaluated loci organized by species and functional category, including but not limited to:
(a) For wheat applications: loci associated with drought tolerance (e.g., loci on wheat chromosome groups 4 and 7 associated with root architecture and osmotic adjustment); disease resistance loci including Lr, Sr, and Yr gene series for leaf rust, stem rust, and stripe rust resistance, respectively; yield component loci associated with thousand kernel weight, grain number per spike, and harvest index; and grain quality loci including Glu-1 and Glu-3 loci encoding high- and low-molecular-weight glutenin subunits. Markers for wheat applications are obtainable from publicly available resources including the GrainGenes database (accessible at wheat.pw.usda.gov) and the International Wheat Genome Sequencing Consortium (IWGSC) reference sequence;
(b) For maize applications: loci associated with drought tolerance, nitrogen use efficiency, disease resistance (e.g., resistance to northern corn leaf blight, gray leaf spot), and yield components. Markers are obtainable from MaizeGDB (accessible at maizegdb.org);
(c) For soybean applications: loci associated with seed composition (protein and oil content), disease resistance (e.g., resistance to soybean cyst nematode, Phytophthora root rot), and maturity group determination. Markers are obtainable from SoyBase (accessible at soybase.org);
(d) For pulse crop applications: loci associated with drought tolerance, yield components (seed size, pod number), disease resistance (fungal or viral pathogens), flowering time, and maturity. Markers are obtainable from KnowPulse (see Sanderson et al., Front. Plant Sci. 10:965, 2019).

### Data Structures Optimization

The system's database is structured to manage and integrate genetic information efficiently. The primary tables include Subjects, Genetic_Profiles, Trait Definitions, and Match_Results. The Subjects table stores information about donors, recipients, or plant specimens with fields for unique identifiers, names or designations, species classifications, birth or germination dates, sex or plant sex types, ethnic backgrounds or varieties, and relevant health or phenotypic information.

The Genetic_Profiles table contains detailed genetic data for each subject, including unique profile identifiers, links to the Subjects table, raw or processed genetic sequence information, lists of identified dominant and recessive traits, HLA types for humans and relevant animals, and specific genetic markers of interest such as SNPs and STRs.

The Trait_Definitions table defines traits and their genetic underpinnings with fields for unique trait identifiers, common trait names, detailed descriptions, dominant or recessive classifications, associated genes, and details about genetic markers linked to each trait. The Match Results table records outcomes of genetic compatibility assessments, storing compatibility scores, shared dominant traits, potential recessive risks, and recommendations.

To optimize data retrieval performance, the system implements specialized indexing structures for genetic marker data. These include B-tree indices optimized for range queries on sequence positions, hash-based indices for rapid exact matching of genetic markers, and specialized bitmap indices for efficient filtering based on trait presence or absence. These indexing structures are dynamically optimized based on observed query patterns to maintain optimal performance as the database grows.

### Genetic Compatibility Assessment Engine

### Match Scoring Module

The match scoring module employs weighted comparison techniques to calculate genetic compatibility scores. The genetic compatibility assessment does not attempt to eliminate the fundamental role of chance in reproduction, but rather provides a framework for identifying and mitigating specific genetic risks while preserving genetic diversity. This approach acknowledges that genetic optimization involves a balance between directed selection and the natural variability that chance introduces into genetic recombination, which is essential for long-term population resilience. This approach assigns different levels of importance to various genetic markers based on their impact on traits of interest, reliability of association, effect size, and relevance to specific objectives. The weighted comparison allows for more nuanced assessment of genetic compatibility by reflecting the relative significance of different genetic features.

The implementation includes algorithms for optimizing weights based on desired outcomes, such as maximizing health-related traits in humans, productivity traits in livestock, or yield characteristics in plants. These algorithms can dynamically adjust weights based on user-defined priorities or population-specific considerations.

The match scoring process generates a compatibility score that represents the overall genetic compatibility between two subjects. This score incorporates measures of genetic distance (the degree of genetic differentiation between subjects), complementary genetic markers (combinations that produce favorable outcomes), and potential genetic risks. The score is presented along with detailed information about shared dominant traits, potential recessive risks, and specific compatibility factors to support informed decision-making.

### Statistical Probability Models Implementation

The genetic compatibility assessment engine incorporates statistical probability models to evaluate the likelihood of specific genetic outcomes based on identified genetic markers and their known inheritance patterns. These models employ Bayesian statistics, logistic regression, Monte Carlo simulations, and Markov models to predict trait expression, disease risk, and other genetic outcomes.

In an embodiment, Bayesian inference is used to estimate posterior distributions of additive genetic effects, dominance effects, penetrance parameters, and other model coefficients. These posterior distributions may be sampled to propagate parameter uncertainty into downstream predictive calculations.

The implementation includes a risk assessment module that calculates the probability of offspring inheriting specific genetic traits or disorders. This module analyzes the carrier status of potential parents and applies Mendelian inheritance principles to determine risk probabilities. For recessive disorders, the system evaluates whether both parents carry recessive alleles and calculates the likelihood of offspring receiving both copies.

In an embodiment, offspring genotype probability distributions are computed at one or more loci based on parental genotype data, including cases where parental genotypes are represented probabilistically. These locus-level genotype probabilities may be aggregated across loci to generate joint or trait-level predictive distributions.

The statistical models account for factors such as penetrance (the proportion of subjects with a genotype who manifest the associated phenotype), expressivity (the degree to which a genotype is expressed in the phenotype), and genetic interactions that may modify inheritance patterns. The models are continuously refined based on new genetic data and observed outcomes to improve prediction accuracy over time.

In an embodiment, dominance effects and other non-additive genetic interactions are explicitly modeled by incorporating genotype interaction terms or dominance encoding schemes. Posterior predictive distributions of phenotypic outcomes may be generated using Monte Carlo simulation, wherein offspring genotypes and model parameters are sampled iteratively to estimate outcome distributions, including expected values, variances, quantiles, and tail-risk probabilities.

In an embodiment, for mate selection applications, the statistical models may incorporate additional parameters related to genetic compatibility between potential partners. These models calculate the probability of offspring inheriting specific combinations of alleles from both parents and predict phenotypic outcomes based on known gene-trait associations. In an embodiment, the implementation includes specialized algorithms for predicting compatibility at immune system loci, where particular combinations may influence reproductive success. The statistical models also evaluate the potential for genetic complementation, where favorable alleles from one parent can compensate for less favorable alleles from the other parent, resulting in offspring with improved genetic fitness compared to either parent alone.

In an embodiment, distributional outputs from Monte Carlo simulation are used to compute compatibility scores or objective functions for ranking or selecting mating pairs. In an embodiment, these objective functions may incorporate one or more of expected phenotypic value; probability of exceeding or falling below defined thresholds; probability of deleterious homozygosity; expected homozygosity; dominance adjusted trait predictions; and risk-adjusted performance metrics.

In an embodiment, compatibility scores may be used within a constrained optimization framework to generate pairwise rankings and/or cohort-level mating assignments subject to defined breeding, inventory, or other risk constraints.

### Cross-Species Adaptation Implementation

The system implements a unified cross-species application paradigm that enables genetic analysis and optimization across humans, animals, and plants while accommodating species-specific biological variations and optimization objectives. This paradigm is built upon a core set of genetic analysis algorithms that operate on fundamental genetic principles common across species, supplemented by specialized modules that address unique aspects of each application domain.

For human applications, the system prioritizes health-related genetic compatibility assessment, focusing on minimizing hereditary disease risk while respecting ethical considerations and regulatory requirements. The human application modules incorporate extensive safeguards to ensure privacy, confidentiality, and appropriate use of genetic information.

Animal applications emphasize health-related genetic compatibility assessment, hereditary disease risk minimization, genetic diversity management, and trait optimization for animal health, productivity, or conservation objectives. These modules incorporate species-specific genetic markers associated with health outcomes, disease resistance, and economically or ecologically valuable traits and implementing algorithms that balance short-term trait optimization with long-term genetic diversity preservation.

Plant applications focus on yield improvement, disease resistance, and environmental adaptability. These modules address the unique genetic characteristics of plant species, which can include polyploidy, asexual reproduction mechanisms, and species-specific genetic elements that influence agricultural productivity or ecological fitness.

The cross-species adaptation is achieved through a layer of abstraction that translates species-specific genetic concepts into standardized representations that can be processed by the core analytical engines. This approach enables knowledge transfer across application domains, allowing innovations in one area to benefit others while maintaining the specialized capabilities required for each domain's unique requirements.

In an embodiment, the cross-species orthologous gene mapping is executed by a hardware-accelerated comparative genomics module utilizing a multi-stage computational pipeline. First, the module implements a Reciprocal Best Hit (RBH) algorithm, wherein nucleotide or translated protein sequences from a first species are queried against a reference genome of a second species using sequence alignment heuristics, and the top-scoring alignments are reciprocally queried back to the first species' genome to mathematically confirm orthology. In a further aspect, to account for evolutionary divergence, the abstraction layer utilizes profile Hidden Markov Models (HMMs) trained on multiple sequence alignments of conserved functional domains. These HMMs calculate emission and transition probabilities to identify remote orthologs that lack high primary sequence identity but maintain structural and functional equivalence. In an embodiment, the mapping further incorporates synteny block analysis to validate orthologous relationships by algorithmically confirming the conservation of gene order and flanking regulatory regions across evolutionary lineages. These algorithmically validated ortholog mappings are stored in a centralized graph database, enabling the genetic compatibility assessment engine to seamlessly substitute a predictive genetic marker established in a model organism for its identified ortholog in a target species during the evaluation of cross-species compatibility scores.

### AI-Based Genetic Optimization

This component serves as a comprehensive AI-driven system that leverages genomic data, bioinformatics, and machine learning to guide fertility decisions across species. It analyzes prospective parents' DNA genetic profiles to recommend optimal matches and select the healthiest offspring matches. By integrating genetic profiling with AI algorithms, it optimizes gamete selection, predicts offspring viability based on genetic compatibility analysis, and flags hereditary disease risks.

The AI component performs comprehensive genetic profiling by sequencing and analyzing DNA from parents (or gamete donors). It identifies key markers including gene variants, chromosomal abnormalities, and other genetic elements, while assessing dominant and recessive traits, disease carrier status, and compatibility at crucial loci. This detailed genetic analysis forms the foundation for all subsequent AI-driven decision processes.

For AI-powered decision support, the system employs proprietary machine learning models to evaluate genetic data with high precision. It implements sophisticated decision-tree algorithms and neural networks that rank potential pairings by predicted health outcomes and compatibility. These models are capable of predicting reproductive outcome likelihood and genetic disorder risks based on patterns learned from large training datasets, enabling data-driven decisions that significantly improve upon traditional methods.

For hereditary risk mitigation, the system flags potentially risky genetic combinations with high sensitivity. It identifies situations where both parents carry recessive disease genes and calculates the probability of genetic disease transmission to offspring using sophisticated statistical models. This proactive approach to genetic risk assessment allows for informed decisions that minimize hereditary disease risks while maximizing positive trait expression.

The implementation of this AI component integrates directly with the hardware-accelerated genetic analysis engine, leveraging the FPGA-based acceleration to process large genetic datasets with significantly improved performance compared to software-only approaches. This hardware-software integration enables real-time analysis of complex genetic data, providing actionable insights at clinically relevant speeds.

### DNA Differences Between Somatic and Gamete Cells

The genetic optimization system acknowledges and accounts for important DNA differences between somatic and gamete cells that impact reproductive outcomes. Somatic and germline cells display distinct mutational landscapes, with germline cells typically having lower mutation rates due to enhanced DNA repair mechanisms and reduced cell division in structures like basal spermatogonia. These differences are critical for maintaining genetic stability across generations. Additionally, epigenetic modifications such as DNA methylation and histone post-translational modifications undergo dynamic changes during gametogenesis, establishing sex-specific methylation patterns that ensure proper gene expression in the germline. In an embodiment, environmental and lifestyle factors can induce epigenetic changes in germ cells that may transmit to subsequent generations as “epimutations,” affecting methylation of DNA and expression of non-coding RNAs in sperm and oocytes. The system incorporates analysis of these somatic-germline differences, accounting for the role of DNA methylation in germline mutation rates, particularly at CpG sites where methylation correlates with higher rates of C>T mutations. This comprehensive approach to genetic analysis enables more accurate assessment of hereditary risk factors and genetic compatibility by distinguishing between somatic mutations and heritable genetic variations.

### Computing Environment and Hardware Acceleration

In an embodiment, the probabilistic modeling, genetic compatibility assessments, and combinatorial optimizations described herein may be executed by a computing device comprising at least one multi-core central processing unit (CPU), at least 64 GB of random-access memory (RAM), and at least one FPGA accelerator card communicatively coupled to the CPU via a Peripheral Component Interconnect Express (PCIe) interface. Exemplary FPGA-based genomic sequence processing architectures and hardware-accelerated mapping systems suitable for use with the present system are described in U.S. Pat. Nos. 9,679,104; 9,014,989; 9,734,284, 9,792,405; 10,847,251; 11,328,793, 12,374,427; and International Patent Publication No. WO 2020/217200, each of which is hereby incorporated by reference in its entirety. In an embodiment, each Monte Carlo iteration may be assigned to a separate processing element within an FPGA systolic array, with offspring genotype sampling, parameter sampling from posterior distributions, and phenotype computation executing in a highly parallelized architecture to minimize clock cycles per iteration.

In an embodiment, the hardware acceleration layer comprises one or more Field-Programmable Gate Arrays (FPGAs), Application-Specific Integrated Circuits (ASICs), or Graphics Processing Units (GPUs). In an embodiment, for example, sequence alignment and variant calling algorithms (such as the Burrows-Wheeler Transform) or Markov chain Monte Carlo (MCMC) sampling tasks may be highly parallelized and offloaded from a central processing unit (CPU) to an FPGA configured with a custom logic circuit specifically designed to execute genomic hashing and probability matrix multiplications. This hardware-software integration provides a specific, tangible improvement to the functioning of the computer itself, drastically reducing the temporal and energetic costs of cohort-level genetic assignment.

Representative FPGAs of this disclosure include the AMD Ultrascale+™ products, including the Spartan UltraScale+, Artix UltraScale+, Kintex UltraScale+, Kintex UltraScale+Gen2, and Virtex UltraScale+; Altera Agilex (9, 7, 5, 3), Stratix 10, Arria 10, and Cyclone series; MicroChip Technology PolarFire family (mid-range FPGAs and SoC FPGAs), IGLOO2, and SmartFusion2; and Lattice Semiconductor CrossLink, ECP, and MachXO products. It is understood that an FPGA will be part of a computer circuit which is typically connected by a circuit board which is configured to be part of a computer system. In some embodiments, the computer system is a computer system described herein.

In some embodiments, this disclosure provides a computer or computer-readable medium with instructions for selecting one or a plurality of genetic traits for breeding optimization. The computer or computer-readable medium includes one or more computer codes or algorithms that perform one or more of the following functions: receive genetic sequence data using a data acquisition module; identify genetic markers within the received genetic sequence data using parallel processing architecture using a hardware-accelerated sequence alignment processor; classify identified genetic markers as dominant or recessive traits based on predefined genetic inheritance models using a trait analyzer; performing a genetic compatibility assessment using a genetic compatibility assessment engine which comprises (i) a match scoring module configured to calculate genetic compatibility scores using weighted comparison of identified dominant and recessive traits; (ii) a risk assessment module configured to evaluate genetic compatibility risks using statistical probability models; and (iii) a database management system configured to store genetic profile data using data structures optimized for genetic sequence retrieval and maintain genetic marker indices optimized for accelerated data retrieval. In some embodiments, the computer or computer-readable medium is further configured to receive inputs of a reference DNA sequence or fragment thereof and align a first sequence and a second sequence with a reference sequence. Software of this disclosure may also provide any of the other logical operations described herein.

In one embodiment, the computer inputs the sequence of a DNA sequence, for example a reference sequence. The reference sequence can be input from any of a number of sources, including experimentally generated data (e.g., sequencing data), data originating from previous recombination products, public and/or commercial databases

The initial sequencing process performed at the laboratory may acquire a substantial amount of genetic data. This amount of genetic data does not need to correspond with the scope of an initial test that caused the sample to be sent to the laboratory. For example, even though the initial test may only consider a small portion of a gene, the laboratory may sequence the entire gene, the entire exome, the entire exome and selected additional regions, or the entire genome of the patient. The genetic data acquired during sequencing may be formatted in a FASTQ format.

In some embodiments, the methods of this disclosure involve software programs that perform bioinformatic operations, such as sequence alignment, variant calling, haplotype calling, and/or imputation for genetic data. Other analytical tools may be used for calling ancestry of the patient. One example of a tool that may be used includes a Burrows-Wheeler Aligner (BWA) process to map low-divergent sequences (e.g., in a FASTQ format generated by a sequencing machine) against a large reference genome, such as a human genome reported in a Binary Alignment Map (BAM) file. Another example of a tool that may be used includes the Genome Analysis Toolkit (GATK) from the Broad Institute in order to perform variant calling. To illustrate, the tool may receive a BAM file and perform variant calling using GATK or a derivative thereof, resulting in a Variant Call Format (VCF) file. In some scenarios, the analytical tools utilize pipelines implementing BWA and GATK processes, such as pipelines developed by Sentieon, Inc. The analytical tools may be machine learning models that are re-trained or altered over time.

In an embodiment, the system may further operate within a cloud-computing or distributed network environment, wherein genomic databases are maintained on secure remote servers and accessed via Application Programming Interfaces (APIs). In an embodiment, the machine learning and AI components (e.g., the Bayesian statistical models) may be continuously trained and updated using federated learning techniques across multiple breeding programs or fertility clinics without exchanging raw underlying genetic data.

### Data Security and Privacy Architectures

In implementations involving human or sensitive elite agricultural genetic data, the database management system and application interfaces are configured to execute privacy-preserving computational protocols. In an embodiment, genomic data and phenotypic records may be de-identified, anonymized, or pseudonymized prior to processing by the compatibility assessment engine.

In an embodiment, the system encrypts genetic data at rest and in transit utilizing advanced encryption standards (e.g., AES-256). For human fertility applications, the system architecture is configured to maintain compliance with relevant data protection and health privacy frameworks, such as the Health Insurance Portability and Accountability Act (HIPAA) or the General Data Protection Regulation (GDPR). In an aspect, the computing device may employ homomorphic encryption, allowing the probabilistic genetic compatibility assessments to be performed on encrypted donor and recipient genetic data without ever decrypting the raw sequence reads in the active memory, thereby preventing unauthorized exposure of sensitive hereditary risk information.

### EXAMPLE IMPLEMENTATIONS

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1 Review of the Field of Genetic Optimization

In an implementation for human fertility enhancement, the system processes genetic data from several (e.g., 50) potential sperm donors and several (e.g. 20) recipient candidates. Genetic samples are collected and DNA extracted using standard protocols. The samples undergo whole-exome sequencing or whole genome, generating, for example, approximately 60-150 GB of raw sequence data per subject.

The exploration of genetic optimization to enhance genetic compatibility across humans involves a multifaceted understanding of genetic compatibility, sexual selection, and gene regulation. Genetic compatibility, as discussed by Puurtinen et al. (Puurtinen, Mikael et al. “The good-genes and compatible-genes benefits of mate choice.” The American naturalist vol. 174,5 (2009): 741-52. doi: 10.1086/606024), is not merely about genetic dissimilarity but rather how well parental genes function together in offspring, which is a crucial aspect of sexual selection beyond the pursuit of ‘good genes” . . . . Tregenza and Wedell (Tregenza T, Wedell N. Genetic compatibility, mate choice and patterns of parentage: invited review. Mol Ecol. 2000; 9(8): 1013-1027. doi: 10.1046/j.1365-294x.2000.00964.x) further elaborate that mate choice for genetic compatibility can drive evolutionary dynamics, although the mechanisms remain largely unknown, with limited evidence of mate choice driven by genetic compatibility in humans. The compatibility of genetic elements is also explored at the molecular level, where Bergman et al. describe the compatibility logic of human enhancer and promoter sequences, suggesting a multiplicative model of gene transcription regulation, which informs understanding of genetic compatibility at a biochemical level (Bergman, Drew T et al. “Compatibility rules of human enhancer and promoter sequences.” Nature vol. 607,7917 (2022): 176-184. doi: 10.1038/s41586-022-04877-w). Additionally, pre-implantation genetic technologies, as reviewed by Scapin et al., allow for the assessment of genetic compatibility in embryos, providing a practical application of genetic compatibility in assisted reproduction (Scapin et al., 2021). The concept of genetic compatibility extends to the immune system, as highlighted by Davis, where specific genes play a significant role in subject health and relationships, potentially influencing mate selection based on genetic compatibility Davis, Daniel M. The Compatibility Gene: How Our Bodies Fight Disease, Attract Others, and Define Our Selves. Oxford University Press, 2013. ISBN: 978-0-19-931641-0.). Furthermore, Harper et al. discuss sexually antagonistic polymorphisms, which may affect genetic compatibility by maintaining alleles that have different effects on disease risk between sexes, thus influencing evolutionary fitness and mate selection (Harper, Jon Alexander et al. “Systematic review reveals multiple sexually antagonistic polymorphisms affecting human disease and complex traits.” Evolution vol. 75,12 (2021): 3087-3097. doi: 10.1111/evo.14394. PMID: 34723381.). Finally, the ethical and societal implications of genetic enhancement, as discussed by Almeida and Diogo, underscore the need for careful consideration of genetic interventions aimed at enhancing human compatibility, balancing therapeutic and enhancement goals within an evolutionary framework (Almeida, Mara, and Rui Diogo. “Human enhancement: Genetic engineering and evolution.” Evolution, Medicine, and Public Health vol. 2019,1 (2019): 183-189. doi: 10.1093/emph/eoz026. PMID: 31620286.). Together, these studies provide a comprehensive view of genetic compatibility, from molecular mechanisms to evolutionary and ethical considerations, highlighting the complexity and potential of genetic optimization in humans.

Genetic optimization across animals to enhance genetic compatibility involves a multifaceted approach that integrates understanding of genetic distance, mate choice, and genomic selection. The concept of optimal mating distance (OMD) suggests that the fitness of offspring is maximized when the genetic distance between parents is neither too small nor too large, balancing the benefits of heterosis with the risks of genetic incompatibility (Wei, Xinzhu, and Jianzhi Zhang. “The optimal mating distance resulting from heterosis and genetic incompatibility.” Science Advances vol. 4,11 (2018): eaau5518. doi: 10.1126/sciadv.aau5518. PMID: 30417098.). This principle is crucial in breeding programs, where genomic selection models are employed to predict crossbred performance, taking into account genetic correlations and the genetic distance between parental lines (Duenk, Pascal et al. “Review: optimizing genomic selection for crossbred performance by model improvement and data collection.” Journal of Animal Science vol. 99 (2021): skab205. doi: 10.1093/jas/skab205.). Mate choice for genetic compatibility, particularly in species like the house mouse, is influenced by genetic elements such as the t haplotype, which can impose significant costs on reproductive success if not properly managed (Lindholm, Anna K. et al. “Mate choice for genetic compatibility in the house mouse.” Ecology and Evolution vol. 3,5 (2013): 1231-1247. doi: 10.1002/ece3.534.). This highlights the importance of genetic compatibility in mate selection, where females may prefer genetically dissimilar males to enhance offspring fitness, although this is not always straightforward due to the complexity of genetic interactions (Piálek, Jaroslav, and Tomáš Albrecht. “Choosing mates: complementary versus compatible genes.” Trends in Ecology & Evolution vol. 20,2 (2005): 63-64. doi: 10.1016/j.tree.2004.12.003.; Puurtinen, Mikael et al. “Mate choice for genetic quality—benefits for both sexes.” Evolution vol. 59,11 (2005): 2400-2405). In insects, engineered genetic incompatibilities (EGIs) have been developed to control population dynamics and prevent gene flow between wild and modified populations, demonstrating a practical application of genetic optimization to manage genetic compatibility (Engineering multiple species-like genetic incompatibilities in insects.” Nature Communications vol. 11,1 (2020): 4468. doi: 10.1038/s41467-020-18348-1. PMID: 32901019). Furthermore, genomic coancestry matrices are used to maintain genetic diversity at specific genomic regions, which is essential for optimizing genetic contributions and controlling inbreeding in breeding programs (Gómez-Romano, Fernando et al. “The use of genomic coancestry matrices in the optimisation of contributions to maintain genetic diversity at specific regions of the genome.” Genetics Selection Evolution vol. 48 (2016): 2. doi: 10.1186/s12711-015-0172-y.). These strategies collectively underscore the importance of genetic compatibility in enhancing reproductive success and maintaining genetic diversity across animal populations, with implications for conservation, agriculture, and the management of wild populations (Wei & Zhang, 2018; Duenk et al., 2021; Gómez-Romano et al., 2016).

Genetic optimization across plants to enhance genetic compatibility involves a multifaceted approach that integrates traditional breeding techniques, genome editing, and an understanding of genetic compatibility frameworks. The exploration of incompatible crosses in plants, as discussed by Visarada et al. (Visarada, Kurella B. R. S. et al. “Exploration of incompatible crosses in plants for novel and useful variations.” Plant Breeding vol. 141,5 (2022): 599-608. doi: 10.1111/pbr.13047), highlights the potential of crossing distant species to introduce novel genetic variations that are heritable and economically beneficial, despite initial appearances of F1 hybrids resembling maternal parents (B. R. S. et al., 2022). This approach is complemented by genome editing tools, which allow for targeted manipulation of plant genomes to increase genetic variation and improve breeding success, as noted by Schleif et al. (Schleif, Nathaniel, Shawn M. Kaeppler, and Heidi F. Kaeppler. “Generating novel plant genetic variation via genome editing to escape the breeding lottery.” In Vitro Cellular & Developmental Biology—Plant vol. 57,4 (2021): 627-644. doi: 10.1007/s11627-021-10213-0). These tools facilitate the introgression of wild accessions and enhance recombination, providing new avenues for plant breeding (Schleif et al., 2021). The concept of genomic compatibility, as explored by Willi and Van Buskirk (Willi, Yvonne, and Josh Van Buskirk. “Genomic compatibility occurs over a wide range of parental genetic similarity in an outcrossing plant.” Proceedings of the Royal Society B: Biological Sciences vol. 272,1570 (2005): 1333-1338. doi: 10.1098/rspb.2005.3077. PMID: 16006327), reveals a quadratic relationship between genetic similarity and offspring performance, suggesting that optimal genetic compatibility spans a broad range of genetic distances, which is crucial for the evolution of mating systems (Willi & Buskirk, 2005). Furthermore, García et al. (García, A. V. et al. “Toward the development of a cross-compatibility framework to enhance the utilization of peanut CWRs.” Crop Science vol. 64,6 (2024). doi: 10.1002/csc2.21332) emphasize the importance of understanding cross-compatibility in utilizing crop wild relatives (CWRs) for breeding peanuts, noting that phylogenetic distance affects hybridization success and pollen viability, which are critical for developing new compatible varieties (García et al., 2024). The molecular basis of self-incompatibility, as reviewed by Yashaswini et al., underscores the role of genetic mechanisms in preventing self-fertilization and promoting outcrossing, which is vital for maintaining genetic diversity and enhancing compatibility in breeding programs (R. et al., 2024). Additionally, the use of genomic selection (GS) and marker-assisted selection, as discussed by Merrick et al. (Merrick, Lance F. et al. “Optimizing Plant Breeding Programs for Genomic Selection.” Agronomy vol. 12,3 (2022): 714. doi: 10.3390/agronomy12030714), provides a framework for optimizing breeding programs by leveraging phenotypic and genotypic data to maximize genetic gain and selection accuracy (Merrick et al., 2022). Finally, the potential for hybridization and transgene escape within Brassica and allied genera, as evaluated by FitzJohn et al. (FitzJohn, Richard G. et al. “Hybridisation within Brassica and allied genera: evaluation of potential for transgene escape.” Euphytica vol. 158,1-2 (2007): 209-230. doi: 10.1007/s10681-007-9444-0), highlights the need for comprehensive risk assessments to ensure compatibility and prevent unintended gene flow (FitzJohn et al., 2007). Collectively, these studies illustrate the complexity and potential of genetic optimization strategies in enhancing plant genetic compatibility, which is essential for sustainable crop improvement and biodiversity conservation.

The concept of “complementary genetic profiles” in the context of dominant versus recessive traits involves understanding how genetic variants interact to express certain phenotypes. Dominant traits are typically expressed when at least one allele is present, whereas recessive traits require two copies of the allele for expression. The study of complementary genetic profiles often involves identifying sets of genetic variants that collectively predict phenotypes, as seen in the Macarons algorithm, which selects complementary subsets of variants to predict quantitative phenotypes without redundancy (Yilmaz, Serhan et al. “Uncovering complementary sets of variants for predicting quantitative phenotypes.” Bioinformatics vol. 38,4 (2022): 908-917. doi: 10.1093/bioinformatics/btab803. PMID: 34864867.). Mendel's foundational work on dominant and recessive traits, such as the 3:1 ratio in hybrid plants, remains crucial for understanding monogenic disorders and their inheritance patterns (Tory, Kálmán. “The dominant findings of a recessive man: from Mendel's kid pea to kidney.” Pediatric nephrology (Berlin, Germany) vol. 39,7 (2024): 2049-2059. doi: 10.1007/s00467-023-06238-9). In the realm of pigmentation traits, genetic prediction models have been developed to predict phenotypes like hair and eye color, with dominant traits often being more straightforward to predict due to their expression with a single allele (Chen, Yi et al. “Genetic prediction of human pigmentation traits.” Forensic Science International: Genetics (2021).). The study of hair-related phenotypes has shown that certain SNPs can predict traits with moderate accuracy, indicating a complex interplay of genetic factors, including dominance and pleiotropy (Pośpiech, Ewelina et al. Human Genetics or Forensic Science International: Genetics (2022)). Furthermore, the concept of genetic dominance is explored through models that reconcile different views on how dominance emerges, suggesting that it is an outcome of complex gene expression and developmental processes (Billiard, Sylvain et al. “The integrative biology of genetic dominance.” Biological Reviews vol. 96,6 (2021): 2925-2942. doi: 10.1111/brv.12786. PMID: 34382317.). In rice, for example, the purple pericarp color is controlled by two dominant complementary genes, Pb and Pp, demonstrating how dominant alleles can determine phenotypic expression (.et al., 2013). Additionally, the concept of complementation in diploid genomes, where recessive mutations do not affect the phenotype unless both alleles are mutated, highlights the importance of understanding genetic interactions in predicting phenotypic outcomes (Stauffer & Cebrat, 2010). Overall, the integration of these studies underscores the complexity of genetic prediction and the necessity of considering both dominant and recessive traits in the context of complementary genetic profiles.

Despite these advances, existing technologies fail to combine genetic profiling, AI-based algorithms, and application-specific optimizations into a unified platform. This invention seeks to address these gaps. The disclosure further incorporates critical understanding about biological differences between somatic and gamete cells that fundamentally impact genetic optimization strategies. Advanced genetic screening methods have revealed distinct mutational landscapes between these cell types that must be considered for accurate genetic compatibility assessment. In somatic cells, mutations accumulate through environmental exposures, replication errors, and endogenous processes, creating mosaicism across tissues. Studies have identified characteristic mutational signatures (such as SBS1 and SBS5/40) which contribute differently to genetic profiles between somatic and germline cells.

In germline cells, particularly spermatogonia, the mutation rate is substantially lower compared to somatic cells, attributed to reduced cell division frequency in basal spermatogonia and enhanced DNA repair mechanisms in these cells. This difference is important because the lower mutation rate in germline cells helps maintain genetic stability across generations, while mutations in these cells can have heritable effects on offspring. In an embodiment, the system may analyze these mutation rate differences when assessing genetic compatibility to better predict hereditary outcomes.

Epigenetic modifications play equally important roles in genetic optimization approaches. DNA methylation undergoes dynamic changes during gametogenesis, with global demethylation in primordial germ cells (PGCs) followed by de novo methylation during gamete maturation. These processes establish sex-specific methylation patterns that ensure proper gene expression in the germline. In an embodiment, the analytical engine disclosed herein may evaluate these methylation patterns as they directly contribute to cellular identity, disease susceptibility, and hereditary trait expression.

Histone modifications, including, for example, H3K36me2/me3 and H3K4me3, regulate gene expression and chromatin structure in germ cells, with H3K4me3 associated with active transcription and H3K27me3 linked to gene repression. The interplay between these histone marks and DNA methylation maintains the balance between pluripotency and differentiation in germ cells. In an embodiment, the system disclosed herein may account for these epigenetic mechanisms as they influence genetic stability and expression patterns across generations.

Environmental and lifestyle factors significantly impact germline epigenetics, with factors such as diet, toxin exposure, and stress inducing epigenetic changes in germ cells. These “epimutations” affect DNA methylation and non-coding RNA expression in sperm and oocytes, potentially transmitting to subsequent generations without altering the underlying DNA sequence. In an embodiment, the comprehensive genetic assessment approach disclosed herein may recognize these environmentally induced modifications when evaluating genetic compatibility.

Somatic mutations that occur after fertilization drive cellular diversity and disease risk. While most are neutral, some confer selective advantages leading to clonal expansion or disease development. In an embodiment, the system disclosed herein may distinguish between somatic variations and heritable germline mutations through specialized analysis algorithms, providing more accurate genetic compatibility assessment.

Transgenerational epigenetic inheritance, where DNA methylation patterns and histone modifications escape global erasure during embryonic development, explains the inheritance of certain traits and disease predispositions without changes to the DNA sequence itself. In an embodiment, the system disclosed herein may analyze specific DNA methylation regions (DMRs) in gametes that can persist through development and influence gene expression in offspring.

In an embodiment, the system models the transmission probability of these epimutations and their impact on offspring phenotypes through an epigenome-aware Bayesian framework. The hardware acceleration unit processes epigenetic input data, such as bisulfite sequencing or chromatin immunoprecipitation sequencing (ChIP-seq) data, to quantify methylation percentages at specific DMRs and calculate histone modification enrichment scores. These quantitative epigenetic metrics are incorporated as dynamic covariates within the statistical probability models. Specifically, the system calculates a transgenerational retention probability for each identified epigenetic mark, applying a probabilistic decay function to account for the likelihood of incomplete global erasure during embryonic reprogramming. During the Monte Carlo simulation iterations, the system samples from these retention probability distributions to predict the offspring's epigenetic state at the evaluated loci. The predicted epigenetic state is then mathematically integrated with the offspring's simulated underlying genotype to generate an epigenetically-adjusted phenotypic posterior distribution. In an aspect, this allows the match scoring module to algorithmically penalize candidate pairings that exhibit a high statistically predicted probability of transmitting environmentally-induced deleterious epimutations, thereby optimizing the complementary index beyond standard Mendelian inheritance.

DNA methylation at CpG sites correlates with higher rates of C>T mutations, revealing complex interactions between epigenetic modifications and genetic stability. In an embodiment, regional methylation levels predict mutation frequencies in specific genomic regions, information that the system may incorporate into its genetic compatibility calculations.

Chromatin regulators like, for example, DNMT3B and G9A/GLP silence germline genes in somatic cells, with their dysfunction linked to neurodevelopmental disorders through ectopic germline gene expression. In an embodiment, the system may evaluate these regulatory mechanisms when assessing genetic compatibility and hereditary disease risk.

Advanced sequencing technologies, particularly massively parallel sequencing (MPS), enable identification of germline and somatic mutations with unprecedented precision. In an embodiment, while technological limitations exist in detecting low-abundance mutations, particularly in somatic mosaicism contexts, the system may employ specialized algorithms to address these challenges, enhancing the accuracy of genetic compatibility assessment across applications.

### Example 2 Implementation Protocol

### Step-by-Step Procedures for Each Claimed Function:

### Genetic Profile Analysis

(a) Sample Collection:
   A. Procedure: Collect biological samples (e.g., blood, saliva, tissue) from subjects.
   B. Considerations: Ensure sterile techniques and proper labeling to maintain sample integrity.
(b) DNA Extraction:
   A. Procedure: Utilize extraction kits to isolate DNA from collected samples.
   B. Considerations: Follow manufacturer protocols to achieve high-quality DNA suitable for sequencing.
(c) Library Preparation:
   A. Procedure: Fragment DNA and ligate adapters to prepare for sequencing.
   B. Considerations: Use standardized kits to ensure compatibility with sequencing platforms.
(d) Sequencing:
   A. Procedure: Perform high-throughput sequencing to obtain raw genetic data.
   B. Considerations: Select appropriate sequencing depth based on the application (e.g., whole-genome, exome).
(e) Data Processing:
   A. Procedure: Align sequences to a reference genome and call variants.
   B. Considerations: Employ bioinformatics tools for accurate alignment and variant detection.
(f) Profile Generation:
   A Procedure: Compile identified variants into a comprehensive genetic profile.
   B. Considerations: Include annotations for known phenotypic associations.

### Risk Assessment Calculations

(a) Data Integration:
   A. Procedure: Combine genetic profiles with medical history and family pedigrees.
   B. Considerations: Ensure data privacy and compliance with ethical standards.
(b) Risk Modeling:
   A. Procedure: Apply statistical models to assess the likelihood of hereditary conditions.
   B. Considerations: Utilize polygenic risk scores where applicable.
(c) Report Generation:
   A. Procedure: Produce comprehensive reports detailing subject genetic risks.
   B. Considerations: Present findings in a clear, actionable format for clinicians and patients.

### Trait Optimization Algorithms

(a) Desired Trait Identification:
   A. Procedure: Define traits of interest (e.g., disease resistance, productivity).
   B. Considerations: Consult with stakeholders to align on trait priorities.
(b) Genetic Marker Association:
   A. Procedure: Identify genetic markers linked to desired traits through genome-wide association studies (GWAS).
   B. Considerations: Ensure statistical significance and replication of findings.
(c) Algorithm Development:
   A. Procedure: Develop algorithms that predict optimal genetic combinations for trait expression.
   B. Considerations: Incorporate machine learning techniques to handle complex trait interactions.
(d) Simulation and Validation:
   A. Procedure: Simulate breeding scenarios and validate predictions with empirical data.
   B. Considerations: Iterate models based on validation outcomes to improve accuracy.

### Cross-Species Adaptation Logic

(a) Comparative Genomics:
   A. Procedure: Analyze genetic similarities and differences across species.
   B. Considerations: Focus on conserved genes and pathways relevant to desired traits.
(b) Ortholog Identification:
   A. Procedure: Identify orthologous genes that perform similar functions in different species.
   B. Considerations: Use sequence alignment tools to detect orthologs accurately.
(c) Functional Validation:
   A. Procedure: Experimentally validate the function of orthologous genes in target species.
   B. Considerations: Employ techniques like gene editing and expression analysis.
(d) Adaptation Strategy Development:
   A. Procedure: Formulate strategies to transfer beneficial traits between species.
   B. Considerations: Address species-specific regulatory elements and ethical considerations.

### Example 3 Optimization of a Dairy Beef-On-Dairy Breeding Program

In an implementation for livestock breeding, the system utilizes genotype data from, at a minimum, 3,000 genetic markers to generate mating recommendations between dairy cows (dams) and beef bulls (sires) through (i) estimating offspring genotype probability distributions and (ii) generating mating recommendations by optimizing an objective function across candidate cow-bull pairings. In an embodiment, the method may output pairwise rankings and/or a cohort-level assignment plan.

In an embodiment, the genotype data may be obtained using commercially available low-density SNP arrays, such as panels comprising approximately 2,900 to 7,000 SNPs (e.g., the Illumina BovineLD BeadChip or the Bovine3K BeadChip; see Boichard et al. (2012), PLOS ONE 7 (3): e34130; Wiggans et al. (2012), J. Dairy Sci. 95:1552-1558; and Illumina GoldenGate Bovine3K Genotyping BeadChip Data Sheet (2011), each of which is incorporated herein by reference). The system imputes the low-density genotype data to a higher-density reference panel (e.g., approximately 50,000 SNPs or more) using population-based imputation algorithms such as Beagle or FImpute, achieving high imputation accuracy (see Dassonneville et al. (2012), J. Dairy Sci. 95:4136-4140; He et al. (2018), BMC Genetics 19:56; and Zhang and Druet (2010), J. Dairy Sci. 93:5487-5494, each of which is incorporated herein by reference). The evaluated genetic markers include specific loci associated with calf birth weight (e.g., the NCAPG/LCORL region on BTA6), calving ease (e.g., markers on BTA18), and known deleterious recessive variants, as well as SNP-based systems for predicting bovine traits (see U.S. Pat. Nos. 7,709,206 and 10,779,518, each incorporated herein by reference). Genotype calls may be stored in standard bioinformatics formats such as VCF (Variant Call Format) or PLINK binary format (.bed/.bim/.fam).

In some embodiments, the genotype data can be extracted from whole genome sequencing performed by a DNA sequencer (e.g., Illumina NovaSeq, Ultima, Roche, Complete Genomics, Element Bio). In some embodiments, the genotype data can be extracted from targeted sequencing performed by a DNA sequencer. The target sequence panel can include targets to the genes and/or loci discussed herein.

For example, these genetic markers may comprise Single Nucleotide Polymorphisms (SNPs) physically identifiable via commercial high-density genotyping arrays, such as the Illumina BovineSNP50 BeadChip or the Neogen GeneSeek Genomic Profiler (GGP) Bovine 100K Beadchip. Specific trait-associated markers utilized in beef-on-dairy genomic selection include, but are not limited to: the DGAT1 gene locus, which is associated with milk fat composition in dairy cows and meat/carcass fatness in beef cattle; the CAPN1 gene locus (e.g., located on chromosome 29) for predicting meat tenderness and Warner-Bratzler shear force; and the FASN (fatty acid synthase) and CCDC57 loci for predicting beef fatty-acid composition. Complete catalogs of these specific genetic markers, including their genomic coordinates and phenotypic associations, can be accessed through public external databases such as the Council on Dairy Cattle Breeding (CDCB) reference database (accessible at uscdcb.com).

In an embodiment, the specific trait-associated genetic markers evaluated by the system for beef-on-dairy genomic selection include, but are not limited to: the DGAT1 gene locus (Diacylglycerol O-Acyltransferase 1, located on Bos taurus chromosome 14), which is associated with milk fat composition in dairy cows and carcass fatness in beef cattle; the CAPN1 gene locus (Calpain 1, located on Bos taurus chromosome 29), which is associated with meat tenderness as measured by Warner-Bratzler shear force; the CAST gene locus (Calpastatin), which modulates calpain activity and is associated with post-mortem meat tenderness; the FASN gene locus (Fatty Acid Synthase), which is associated with beef fatty-acid composition and intramuscular fat content; the CCDC57 gene locus, which is associated with beef fatty-acid composition; the MSTN gene locus (Myostatin, also known as GDF8), which is associated with muscle development, double muscling phenotype, and carcass yield; the LEP gene locus (Leptin), which is associated with feed intake regulation, fat deposition, and carcass composition; and the SCD gene locus (Stearoyl-CoA Desaturase), which is associated with fatty acid desaturation and meat quality attributes.

In an embodiment, the system further evaluates markers associated with reproductive and health traits, including: loci associated with bovine respiratory disease (BRD) susceptibility; loci associated with bovine viral diarrhea (BVD) resistance; loci within the bovine Major Histocompatibility Complex (BoLA) associated with immune response and disease resistance; loci associated with parasite resistance; and loci associated with heat tolerance, including variants in the SLICK gene (PRLR locus on chromosome 20). For dairy-specific traits, the system may evaluate markers associated with milk protein variants (e.g., kappa-casein genotype, beta-lactoglobulin genotype), which influence cheese-making properties and milk quality.

In an embodiment, complete catalogs of trait-associated genetic markers for bovine applications, including their genomic coordinates, allele frequencies, and phenotypic effect estimates, are accessible through one or more publicly available external databases including, but not limited to: the Council on Dairy Cattle Breeding (CDCB) reference database (accessible at uscdcb.com); the Animal Quantitative Trait Loci Database (Animal QTLdb, accessible at animalgenome.org/cgi-bin/QTLdb/index), which houses over 277,000 cattle QTL and association data curated from over 1,225 publications (see, e.g., Hu, Z.-L., et al., “Bringing the Animal QTLdb and CorrDB into the future: meeting new challenges and providing updated services,” Nucleic Acids Research, Volume 50, Issue D1, Pages D956-D961 (2022), doi: 10.1093/nar/gkab1116); the Bovine Genome Variation Database (BGVD, see Chen, N., et al., “BGVD: An Integrated Database for Bovine Sequencing Variations and Selective Signatures,” Genomics Proteomics Bioinformatics, 18 (2): 186-193 (April 2020), doi: 10.1016/j.gpb.2019.03.007); the NCBI dbSNP database; and the Ensembl genome browser (accessible at ensembl.org). These databases provide the person of ordinary skill in the art with access to comprehensive lists of validated bovine genetic markers, their chromosomal positions, allelic variants, and trait associations sufficient to practice the current invention.

In an embodiment, the system utilizes genetic marker information obtained from one or more of the following publicly available external databases and resources, which are incorporated herein by reference to the extent necessary to enable the practice of the claimed invention:
(a) The Animal Quantitative Trait Loci Database (Animal QTLdb, accessible at animalgenome.org/cgi-bin/QTLdb/index), which as of its most recent release houses over 277,000 QTL and association records curated from over 2,800 publications across seven livestock species including cattle, pigs, chickens, sheep, horses, rainbow trout, and catfish (see Hu, Z.-L., et al., “Bringing the Animal QTLdb and CorrDB into the future: meeting new challenges and providing updated services,” Nucleic Acids Research, Volume 50, Issue D1, Pages D956-D961 (2022), doi: 10.1093/nar/gkab1116, incorporated herein by reference);
(b) The Bovine Genome Variation Database (BGVD, see Chen, N., et al., “BGVD: An Integrated Database for Bovine Sequencing Variations and Selective Signatures,” Genomics Proteomics Bioinformatics, 18 (2): 186-193 (April 2020), doi 10.1016/j.gpb.2019.03.007, incorporated herein by reference);
(c) The Council on Dairy Cattle Breeding (CDCB) reference database (accessible at uscdcb.com), which provides genomic evaluations for dairy bulls and cows, including predicted transmitting abilities (PTAs) for production, health, fertility, and conformation traits;
(d) The NCBI dbSNP database (accessible at ncbi.nlm.nih.gov/snp), which catalogs single nucleotide polymorphisms and short genetic variations across species;
(e) The Ensembl genome browser (accessible at ensembl.org), which provides genome annotation, variation data, and comparative genomics information for vertebrate genomes including Bos taurus;
(f) The Genome Aggregation Database (gnomAD, accessible at gnomad.broadinstitute.org), which provides allele frequency data and variant annotations across diverse human populations derived from large-scale sequencing projects;
(g) ClinVar (accessible at ncbi.nlm.nih.gov/clinvar), which archives reports of the relationships among human genetic variations and phenotypes with supporting evidence;
(h) The Online Mendelian Inheritance in Man (OMIM) database (accessible at omim.org), which catalogs human genes and genetic phenotypes; and
(i) The NHGRI-EBI GWAS Catalog (accessible at ebi.ac.uk/gwas), which provides a curated collection of published genome-wide association studies.

In an embodiment, the system evaluates genetic markers at a plurality of loci numbering at least 3,000 markers for livestock breeding applications. In an aspect, the minimum of 3,000 markers is selected to provide sufficient genome-wide coverage for accurate estimation of genomic relatedness, prediction of breeding values, and identification of regions of homozygosity and heterozygosity across the bovine genome. In an embodiment, the markers may number approximately 2,900 to 7,000 when obtained from commercially available low-density SNP arrays (e.g., the Illumina BovineLD BeadChip or Bovine3K BeadChip), approximately 50,000 when obtained from medium-density arrays (e.g., the Illumina BovineSNP50 BeadChip), or approximately 100,000 or more when obtained from high-density arrays (e.g., the Neogen GeneSeek Genomic Profiler Bovine 100K BeadChip or the Illumina BovineHD BeadChip comprising approximately 777,000 SNPs).

In an embodiment, the specific genetic markers evaluated by the system for bovine applications are organized by functional category and include, but are not limited to, the following loci and their associated traits:
(a) Production and carcass quality markers: the DGAT1 gene locus (Diacylglycerol O-Acyltransferase 1, located on Bos taurus chromosome 14, BTA14), associated with milk fat composition in dairy cattle and carcass fatness in beef cattle; the CAPN1 gene locus (Calpain 1, located on BTA29), associated with meat tenderness as measured by Warner-Bratzler shear force; the CAST gene locus (Calpastatin), which modulates calpain activity and is associated with post-mortem meat tenderness; the FASN gene locus (Fatty Acid Synthase), associated with beef fatty-acid composition and intramuscular fat content; the CCDC57 gene locus, associated with beef fatty-acid composition; the MSTN gene locus (Myostatin, also known as GDF8), associated with muscle development, double muscling phenotype, and carcass yield; the LEP gene locus (Leptin), associated with feed intake regulation, fat deposition, and carcass composition; the SCD gene locus (Stearoyl-CoA Desaturase), associated with fatty acid desaturation and meat quality attributes; and the NCAPG/LCORL region on BTA6, associated with calf birth weight and growth traits.
(b) Reproductive and calving markers: loci on BTA18 associated with calving ease; loci associated with gestation length, fertility proxies (e.g., conception success, open days, and calving interval), daughter pregnancy rate (DPR), cow conception rate (CCR), and heifer conception rate (HCR); and loci associated with stillbirth risk.
(c) Health and disease resistance markers: loci within the bovine Major Histocompatibility Complex (BoLA) associated with immune response and disease resistance; loci associated with bovine respiratory disease (BRD) susceptibility; loci associated with bovine viral diarrhea (BVD) resistance; loci associated with parasite resistance; loci associated with heat tolerance, including variants in the SLICK gene (PRLR locus on chromosome 20); and loci associated with somatic cell score (SCS) as an indicator of mastitis susceptibility.
(d) Milk quality and composition markers: loci associated with milk protein variants including kappa-casein (CSN3) genotype and beta-lactoglobulin (LGB) genotype, which influence cheese-making properties and milk quality; loci associated with casein gene cluster variants (CSN1S1, CSN1S2, CSN2) on BTA6; and loci associated with fat percentage and protein percentage.
(e) Known deleterious recessive variants: JH1 and JH2 haplotypes in Jersey cattle; HH1 through HH6 haplotypes in Holstein cattle; and analogous deleterious recessive haplotypes identified in Brown Swiss, Ayrshire, and other dairy and beef breeds.

In some embodiments, the genetic markers can include or exclude: those for Milk Production & Quality (LALBA (Alpha-lactalbumin)—Linked to milk production, LGB (Beta-lactoglobulin)-Linked to milk protein composition, KIRREL3, LRRC3, or TSPEAR-Associated with milk fat and yield, particularly on BTA 29 and BTA 1, DGAT1—Known to influence milk fat percentage; Fertility & Reproduction (COQ9—Associated with fertility and reproductive function), HSPA1A/HSPA1L—Linked to heat stress tolerance and embryo survival, Scrotal Circumference (Trait)-Used as a genetic indicator for bull fertility and daughter fertility; Growth & Carcass Quality: LEP (Leptin)—Influences fat metabolism and feed efficiency, QTLs for Body Weight: Regions on chromosomes 1, 4, 14, 22, 26, and 27 affect growth and fat thickness (often studied in Brangus/Angus), Health & Physical Traits: TLR4: Associated with mastitis resistance, Polledness—Genetic testing for hornless cattle, Coat Color: Genes for specific colors (e.g., in Speckle Park).

In some embodiments, the methods of this disclosure can screen for disfavorable genetic traits including CVM (Complex Vertebral Malformation)-a genetic defect often tested in dairy cattle; and Bulldog Syndrome-A lethal genetic condition.

In an embodiment, a breeding population includes at least N dairy cows, where N may range from at least 20, and semen sourced from at least M bulls, where M may range from at least 2 bulls. Each animal is uniquely identified (e.g., ear tag, RFID, or database identifier). The system receives and stores, for each animal, one or more of:
Pedigree data: sire, dam, and lineage identifiers when available
Reproductive outcomes: mating/insemination date, gestation length, stillbirth count, calve weight at birth, calve survival to day 7, day 21, and weaning, open days, and calving interval.
Growth outcomes: calve weights at specified ages (e.g., day 7/14/21/weaning).
Health outcomes: disease events, treatments, and culling reasons.
Genomic inputs: genotype markers from sequenced DNA

In an embodiment, records may be stored in a relational database and linked via the unique animal identifier.

DNA is obtained from tissue, blood, buccal samples, or semen samples using standard protocols. Genotype data are obtained using one or more of SNP arrays, targeted marker panels, low-pass sequencing with imputation, or whole genome sequencing. In an embodiment, when sequence reads are used, reads may be aligned to a Bos taurus reference genome and calling variants at genomic positions. Genotype calls for each animal are stored in digital form.

In an embodiment, genotype markers may include trait-associated SNPs; known deleterious variant loci; loci associated with calf birth weight, calf size, and/or calf survival; loci associated with calving ease, dystocia risk, fertility, and/or gestation length; loci used to estimate genomic relatedness and/or optimal mating distance. Genotype markers are available from publicly available resources such as the Bovine Genome Variation Database (see, e.g., Chen, N., et al., “BGVD: An Integrated Database for Bovine Sequencing Variations and Selective Signatures,” Genomics Proteomics Bioinformatics, 18(2): 186-193 (April 2020), doi 10.1016/j.gpb.2019.03.007; PMID: 32540200). In some embodiments, genotype markers may be those described in the art (Singh, U. et al., Molecular markers and their applications in cattle genetic research: A review, Biomarkers and Genomic Medicine, Volume 6, Issue 2, 2014, Pages 49-58, doi.org/10.1016/j.bgm.2014.03.001; E. W. Brascamp, et al., Economic Appraisal of the Utilization of Genetic Markers in Dairy Cattle Breeding, Journal of Dairy Science, Volume 76, Issue 4, 1993, Pages 1204-1213, doi.org/10.3168/jds.S0022-0302 (93) 77450-0; C. Schrooten, et al., Genetic Progress in Multistage Dairy Cattle Breeding Schemes Using Genetic Markers, Journal of Dairy Science, Volume 88, Issue 4, 2005, Pages 1569-1581, doi.org/10.3168/jds.S0022-0302 (05) 72826-5; Salisu et al., Molecular markers and their Potentials in Animal Breeding and Genetics, Nigerian J. Anim. Sci. 2018, 20 (3): 29-48; Koshchaev, A. G., et al., Allelic Variation of Marker Genes of Hereditary Diseases and Economically Important Traits in Dairy Breeding Cattle Population, Journal of Pharmaceutical Sciences and Research; Cuddalore Vol. 10, Iss. 6, (June 2018): 1566-1572), the contents of each of which is herein incorporated by reference.

In an embodiment, genotype calls may be stored as allele dosage, phased haplotypes, or genotype probabilities. In an embodiment, where genotype uncertainty exists (e.g., imputed genotypes), posterior genotype probabilities may be stored and propagated downstream.

The system performs one or more quality control operations including: removal or imputation of missing genotypes; filtering by minor allele frequency threshold; exclusion of loci with low call rate; optional Hardy-Weinberg filtering for quality control purposes; and pedigree/genotype consistency checks.

Phenotype values are normalized and/or modeled with appropriate link functions such as logistic for survival, ordinal/probit for calving difficulty, and Gaussian for birth weight.

The system estimates parameters for one or more traits using machine learning models that include Bayesian statistical models, logistic regression models, decision tree models, neural network models, and ensemble learning methods. In an embodiment, binary outcomes such as dystocia occurrence, stillbirth risk, or early calf mortality may be modeled using logistic regression or other probabilistic classifiers. In an embodiment, sequential biological outcomes, including survival across development stages, may be modeled using Markov models that estimate transition probabilities between states (e.g., birth to neonatal survival to weaning). Bayesian inference is used to estimate posterior distributions of genetic effect parameters, including additive and dominance contributions.

In an embodiment, prior distributions for additive marker effects follow a mixture of normal distributions (e.g., BayesB) or a scaled inverse chi-squared distribution for variance components to calculate genomic estimated breeding values (GEBVs) (see Meuwissen et al. (2001), Genetics 157:1819-1829; and International Patent Publication No. WO 2015/100236, each of which is incorporated herein by reference). In an embodiment, the number of Markov chain Monte Carlo (MCMC) iterations utilized by the system may range from 10,000 to 500,000 with a burn-in period of 1,000 to 50,000 iterations, and convergence may be assessed using the Gelman-Rubin diagnostic or effective sample size metrics.

For each candidate cow-bull pairing, the system computes an offspring genotype probability distribution at one or more loci. For each locus, given parental genotype probabilities, the system computes the probability that offspring are homozygous recessive, heterozygous, or homozygous dominant for the given locus. If parental genotypes are uncertain, parental genotype posteriors are integrated to compute offspring genotype probabilities.

For known deleterious recessive loci, the system computes the probability of homozygous recessive offspring genotype for each pairing and optionally an aggregated recessive risk score across loci. In an embodiment, pairings exceeding a risk threshold may be excluded or penalized.

The system uses Monte Carlo simulation to propagate genotype uncertainty and Bayesian parameter uncertainty into predicted offspring outcome distributions. For each candidate pairing and for each Monte Carlo iteration, offspring genotypes are sampled at the given locus from the offspring genotype probability distribution, the additive and dominance effect parameters are sampled from the posterior distribution or the breeding values are sampled directly from the posterior distributions, simulated offspring trait outcomes and/or trait risk probabilities for one or more traits are computed, and the simulation outcomes are recorded. For each pairing, the system produces one or more distributional summaries including: posterior predictive mean and variance for each trait, probability of exceeding a threshold, probability of adverse events, and expected and tail risk metrics. In an embodiment, these distributional features may be used directly in scoring and optimization.

In an embodiment, a complementary index is computed for each candidate pairing by combining multiple distribution-derived metrics. In an embodiment, this complementary index may include expected economic value based on predicted calf performance and costs; expected carcass/growth index; penalty terms for dystocia risk and/or stillbirth risk; penalty terms for deleterious homozygosity probability; penalty terms for high relatedness/inbreeding risk; a dominance/heterozygosity benefit term derived from simulated offspring heterozygosity or dominance contribution.

The pairing score may take the form:

Sₚₐᵢᵣ=w₁·E[V]−w₂·P(dystocia)−w₃·P(deleterious homozygosity)−w₄·E[F_{offspring}]+w₅·E[dominance benefit]

where:
E[V] represents expected net value or a multi-trait index derived from posterior predictive outcomes
F_{offspring} represents expected offspring inbreeding/genomic relatedness
the weights w₁-w₅ are user-configurable or automatically tuned.

Candidate pairings are ranked by pairing score, and for each cow, one or more top-ranked bulls are output.

The weights (w₁ through w₅) serve as normalization coefficients that scale disparately measured variables (e.g., continuous genetic variance metrics, binary probability values, and economic dollar values) into a unified, dimensionless objective score. In an embodiment, the weights are dynamically tuned utilizing a machine learning gradient descent algorithm trained on historical herd economic data, wherein the weights are continuously adjusted to maximize the correlation between historical pairing scores and actual realized lifetime profitability of the progeny. In an embodiment, alternatively, the weights may be user-configured via a graphical user interface to reflect specific, localized herd management priorities (e.g., increasing w₂ in facilities with limited veterinary intervention capabilities for dystocia).

In an exemplary embodiment, the weights may be constrained within specific ranges to optimize herd outcomes. In an embodiment, for example, w₁ may range from 0.3 to 0.5, w₂ from 0.1 to 0.3, w₃ from 0.05 to 0.2, w₄ from 0.05 to 0.15, and w₅ from 0.05 to 0.15, where the weights are normalized to sum to 1.0. In an embodiment, these weights may be configured by a user through a graphical interface or determined automatically utilizing machine learning algorithms trained on historical herd performance data. For examples of computational methods for simulating progeny from genome-wide markers and optimizing selection combinations, see U.S. Pat. Nos. 8,874,420; 11,744,199; and U.S. Patent Application Publication Nos. 2016/0309685 and 2007/0105107, each of which is incorporated herein by reference.

A cohort-level assignment is generated by selecting bull assignments for all cows in the herd under one or more constraints, including: a maximum number of inseminations per bull; semen inventory limits; breeding window constraints; herd management constraints such as avoiding excessive concentration of a bull; maximum allowable expected dystocia probability per cohort; and maximum allowable expected recessive risk. In an embodiment, the cohort assignment may be solved using integer programming, mixed-integer programming, evolutionary algorithms, or other combinatorial optimization techniques.

The system outputs a breeding plan comprising the recommended bull assignment per cow, pairing score and component metrics, posterior predictive summaries (mean/variance/quantiles) for one or more traits, probability summaries for adverse events, deleterious locus risk flags and/or thresholds, and optional explanations identifying which components most influenced the score. In an embodiment, outputs may be provided through a GUI, report, API, or integration into herd management software.

The performance of the system is evaluated by comparing predicted and/or observed outcomes against one or more baselines, including: random bull assignment, pedigree-only mating control, top-index bull selection without Monte Carlo or Bayesian uncertainty propagation, or mating selection using mean-only expected values without dominance modeling.

Evaluation metrics include dystocia incidence, stillbirth incidence, calf birth weight distribution, survival to defined time points, realized economic return, and changes in deleterious allele homozygosity rates.

Following the generation of the breeding plan and cohort-level assignment, the system interfaces with herd management hardware to execute the physical breeding process. In an embodiment, the system triggers the sorting and separation of the designated dairy cows into breeding pens and outputs an execution command to a reproductive technician or automated insemination system. The designated cows are then physically inseminated with the selected semen from the assigned beef bulls corresponding to the highest pairing scores (Sₚₐᵢᵣ), thereby physically transforming the algorithmic assignment into the conception and gestation of the probabilistically optimized F1 hybrid offspring.

In an aspect of the dairy beef-on-dairy breeding program implementation, the system validates the accuracy of its probabilistic predictions by comparing simulated offspring outcome distributions against observed phenotypic data from prior breeding cycles. In an embodiment, the system receives feedback data comprising actual calf birth weights, calving ease scores, survival outcomes, and growth performance from offspring produced using prior system-generated mating recommendations. In an embodiment, the system utilizes this feedback data to recalibrate Bayesian model parameters, update posterior distributions of marker effects, and refine the weighting coefficients (w₁ through w₅) of the pairing score objective function. This iterative feedback loop constitutes a machine-learning-driven continuous improvement process that enhances prediction accuracy over successive breeding cycles, providing a concrete technical improvement to the functioning of the breeding optimization system.

In an embodiment, the system implements cross-validation procedures to assess prediction accuracy prior to deployment. The system may perform k-fold cross-validation, in which the genotyped and phenotyped population is partitioned into k subsets, with the model trained on k−1 subsets and prediction accuracy assessed on the withheld subset, iteratively across all partitions. Prediction accuracy may be quantified using one or more metrics including correlation between predicted and observed trait values, mean squared error, area under the receiver operating characteristic curve (AUC-ROC) for binary outcomes (e.g., dystocia, stillbirth), and calibration plots comparing predicted probabilities to observed frequencies.

### Example 4 Probabilistic Donor-Recipient Compatibility Assessment for Human Fertility Applications

In an implementation directed at human fertility applications, the system processes genetic data from sperm donors and recipient candidates. In an embodiment, for example, genotype data may be obtained from 50 donor candidates and 20 recipient candidates, although the system is not limited to these numbers.

DNA is obtained from tissue, blood, buccal samples, or semen samples using standard protocols. In an embodiment, sequencing may be performed using whole-genome sequencing, whole-exome sequencing, or targeted marker panels. Sequenced reads are aligned to a human reference genome (e.g., GRCh38), and variants are called at genomic loci. Genotype calls for each subject are stored in digital form.

The system identifies and calls variants for at least 1,000 loci, including loci associated with monogenic recessive disorders; loci associated with dominant disease risk; loci contributing to polygenic risk scores; loci associated with fertility, metabolic traits, immune compatibility, or other clinically relevant traits; and loci associated with optimal mating distance. In an embodiment, genotype calls may be represented as discrete genotype states or probabilistic genotype distributions where uncertainty exists. Genotype markers are available from databases such as the Genome Aggregation Database (GnomAD) and ClinVar.

In an embodiment, the system evaluates genetic markers obtainable from one or more publicly available databases including, but not limited to: the Genome Aggregation Database (gnomAD, accessible at gnomad.broadinstitute.org), which provides allele frequency data and variant annotations across diverse human populations; ClinVar (accessible at ncbi.nlm.nih.gov/clinvar), which archives reports of the relationships among human variations and phenotypes with supporting evidence; the Online Mendelian Inheritance in Man (OMIM) database (accessible at omim.org), which catalogs human genes and genetic phenotypes; the Human Gene Mutation Database (HGMD); the Pharmacogenomics Knowledge Base (PharmGKB); and the NHGRI-EBI GWAS Catalog (accessible at ebi.ac.uk/gwas), which provides a curated collection of published genome-wide association studies. These databases enable the identification of loci associated with over 6,000 monogenic disorders, polygenic risk loci for common diseases, and pharmacogenomic variants, providing sufficient information for a person of ordinary skill in the art to select appropriate markers for evaluation by the system disclosed herein.

In an embodiment, the system evaluates immune compatibility by assessing genetic variation at Human Leukocyte Antigen (HLA) loci, including HLA-A, HLA-B, HLA-C, HLA-DRB1, HLA-DQB1, and HLA-DPB1. HLA compatibility between reproductive partners may influence reproductive success through mechanisms including natural killer cell-mediated immune tolerance at the maternal-fetal interface. In an embodiment, the system may further evaluate variants in killer-cell immunoglobulin-like receptor (KIR) genes, which interact with HLA ligands to modulate immune responses during implantation and placentation.

The genetic compatibility assessment module applies statistical and machine learning models to estimate the probability of genetic outcomes for potential offspring. The predictive models include Bayesian models, logistic regression models, decision tree models, neural network models, and ensemble models combining multiple predictors. In an embodiment, binary outcomes such as disease presence or absence may be modeled using logistic regression or probabilistic classifiers. Bayesian inference is used to estimate posterior distributions for trait effects, disease risk contributions, and genetic interaction parameters.

For monogenic recessive conditions, the system computes offspring genotype probability distributions for each donor-recipient pairing using Mendelian inheritance principles. Where parental genotype uncertainty exists, posterior genotype probabilities are integrated to determine offspring genotype distributions.

For polygenic traits, the system estimates posterior distributions of additive and, where applicable, dominance effects. Posterior predictive distributions for offspring phenotypes or risk scores are generated by sampling from these posterior parameter distributions.

In an embodiment, the statistical framework may account for penetrance, variable expressivity, gene-gene interactions, and other modifiers of phenotypic expression.

For each donor-recipient pairing, the system performs Monte Carlo simulation to generate offspring genotype and phenotype distributions. For each simulation iteration, offspring genotypes are sampled from one or more loci from parental genotype probability distributions; trait effect parameters are sampled from posterior distributions; and offspring phenotype values and/or disease risk probabilities are computed. After sufficient iterations (e.g., 1,000-100,000 simulations), the system computes distributional summaries including: expected disease risk probability; probability of homozygous recessive genotypes at specified loci; expected polygenic risk score, distribution variance and selected quantiles; and probability of exceeding defined clinical risk thresholds. These distributional outputs provide both expected value and uncertainty information for each pairing.

Compatibility between donor and recipient is determined using a composite objective function derived from distributional outputs rather than solely mean risk values. In an embodiment, the objective function may incorporate one or more of: probability of recessive disorder in offspring; posterior expected polygenic risk score; probability of exceeding defined disease risk thresholds; dominance-adjusted trait predictions; heterozygosity or immune-locus complementarity metrics; and risk penalties associated with high penetrance alleles. In an embodiment, the objective function may be expressed as a weighted combination of distribution-derived metrics. In an embodiment, weights may be user-defined, clinician-configured, or dynamically determined. In an embodiment, pairings may be ranked based on the objective score.

The system outputs for each donor-recipient pairing: the posterior predictive disease risk probabilities; expected and percentile-based trait scores; probability of deleterious homozygous genotype; dominance-adjusted compatibility metrics; and explanation summaries of identifying high-impact loci. In an embodiment, system outputs may be compared against conventional carrier screening and manual genetic counselor analysis for concordance in identifying high-risk pairings. In an embodiment, the automated probabilistic framework reduces analysis time while preserving or improving risk stratification consistency.

### Example 5 Probabilistic Cross Optimization in a Pulse Crop Breeding Program

In an implementation directed at crop improvement, the system is applied to optimize crosses within a pulse crop breeding program, such as lentil (Lens culinaris), chickpea (Cicer arietinum), or pea (Pisum sativum), although the system is not limited to these species or to pulse crops.

Genotype data are obtained from a population of parental lines. In an embodiment, DNA may be extracted from leaf tissue or seed samples using standard protocols and sequenced using whole-genome sequencing, reduced-representation sequencing, or targeted marker panels. In an embodiment, sequence reads are aligned to an appropriate reference genome (e.g., Lcu.2RBY CDC Redberry Lens culinaris genome assembly), and variants are called. In an embodiment, loci where variants are called may comprise: loci associated with drought tolerance; loci associated with yield components (e.g., seed size, pod number); loci associated with disease resistance (e.g., fungal or viral pathogens); loci associated with flowering time or maturity; and loci used for estimating genomic relatedness (see, e.g., Sanderson, L-A., et al., “KnowPulse: A Web-Resource Focused on Diversity Data for Pulse Crop Improvement,” Front. Plant Sci., 10:965 (2019), doi: 10.3389/fpls.2019.00965). Genotype markers are available through publicly available resources such as KnowPulse. Genotypes may be encoded as allele dosage values, haplotypes, or probabilistic genotype calls.

In an embodiment, for crop and plant applications, the system may utilize genetic marker data obtainable from one or more publicly available databases including, but not limited to: KnowPulse (accessible at knowpulse.usask.ca), which provides diversity data for pulse crop improvement (see Sanderson, L-A., et al., “KnowPulse: A Web-Resource Focused on Diversity Data for Pulse Crop Improvement,” Front. Plant Sci., 10:965 (2019), doi: 10.3389/fpls.2019.00965); the Gramene database (accessible at gramene.org), which provides comparative genomics resources for crop and model plant species; the Sol Genomics Network (SGN, accessible at solgenomics.net) for Solanaceae species; the Maize Genetics and Genomics Database (MaizeGDB, accessible at maizegdb.org); the GrainGenes database for small grains including wheat, barley, oats, and rye; the Triticeae Toolbox (T3) for wheat and barley breeding data; SoyBase (accessible at soybase.org) for soybean, the Planteome database (accessible at planteome.org) for plant trait ontologies and phenotype data, and the Phytozome database (accessible at phytozome-next.jgi.doe.gov) for plant comparative genomics. For each target crop species, these databases provide the person of ordinary skill in the art with access to validated genetic markers, their chromosomal positions, and trait associations.

In an embodiment, for wheat applications, genetic markers evaluated by the system may include loci associated with drought tolerance (e.g., loci on wheat chromosome groups 4 and 7 associated with root architecture and osmotic adjustment), disease resistance loci (e.g., Lr, Sr, and Yr gene series for leaf rust, stem rust, and stripe rust resistance, respectively), yield component loci (e.g., loci associated with thousand kernel weight, grain number per spike, and harvest index), and grain quality loci (e.g., Glu-1 and Glu-3 loci encoding high- and low-molecular-weight glutenin subunits). In an embodiment, for maize applications, genetic markers may include loci associated with drought tolerance, nitrogen use efficiency, disease resistance (e.g., resistance to northern corn leaf blight, gray leaf spot), and yield components. For soybean applications, genetic markers may include loci associated with seed composition (protein and oil content), disease resistance (e.g., resistance to soybean cyst nematode, Phytophthora root rot), and maturity group determination.

One or more agronomic traits are modeled using Bayesian statistical methods. For example, in an embodiment, drought tolerance, yield, and disease resistance may be modeled using additive genetic effects; dominance effects (where relevant in hybrid systems); genotype-by-environment interaction terms; and environmental covariates (e.g., rainfall, soil type, temperature). Posterior distributions of marker effects and trait parameters are estimated using Bayesian inference methods such as MCMC or variational inference. In an embodiment, the models may incorporate multi-environment trial data to estimate environment-specific and environment-robust trait effects.

For each potential cross between two parental lines, the system computes offspring genotype probability distributions at one or more loci. For each locus, the system determines: probability of homozygous reference genotype; probability of heterozygous genotype; and probability of homozygous alternate genotype. Where parental genotype uncertainty exists, parental posterior genotype probabilities are integrated into offspring probability calculations. In polygenic trait contexts, expected additive and dominance contributions for offspring are derived from locus-level genotype probabilities and posterior marker effect distributions.

The system performs Monte Carlo simulation for each candidate cross to generate predicted distributions of progeny performance. For each simulation iteration, the offspring genotypes are sampled at one or more loci from the computed offspring genotype probability distributions; trait effect parameters are sampled from posterior distributions; and simulated progeny values are computed (e.g., drought tolerance index, yield score, disease resistance probability). After sufficient iterations (e.g., 1,000-100,000), the system computes the expected progeny mean performance; predicted genetic variance among progeny; probability of exceeding defined breeding thresholds; probability of unfavorable allele fixation; and expected heterozygosity or diversity metrics. These distributional summaries enable evaluation of both expected gain and variability within progeny populations.

The candidate crosses are evaluated using a multi-objective function incorporating distribution-derived metrics. In an embodiment, the objective function may include one or more of expected progeny yield; probability of exceeding drought tolerance threshold; disease resistance probability; maintenance of genetic diversity; reduction in deleterious allele frequency; and predicted segregation variance to maximize selection potential in subsequent generations.

In an embodiment, the objective function may take the form:

S_{cross}=w₁·E[Yield]+w₂·P(Drought≥D₀)−w₃·P(DiseaseSusceptible)+w₄·E[GeneticVariance]−w₅·F_{relatedness}
where weights w₁-w₅ are configurable based on breeding goals.

In an embodiment, rather than ranking subject crosses independently, the system selects a portfolio of crosses to be advanced in a breeding cycle. In an embodiment, the portfolio may incorporate maximum number of crosses per cycle; seed production constraints; desired balance between short-term gain and long-term diversity; and environmental target profiles. In an embodiment, a combinatorial optimization algorithm (e.g., integer programming or evolutionary algorithms) may be used to select a set of crosses that maximizes aggregate breeding objectives across the breeding program.

The system outputs ranked cross recommendations; posterior predictive trait distributions for each cross; probability of achieving defined breeding targets; diversity and relatedness metrics; and risk summaries for deleterious allele combinations. In an embodiment, outputs may be integrated into breeding management software to guide crossing decisions.

Upon selection of the portfolio of crosses utilizing the combinatorial optimization algorithm, the recommended crosses are physically executed. In an embodiment, this comprises physically planting the seeds of the selected parental lines, manually or mechanically emasculating the selected female parental flowers, and applying the pollen from the selected male parental flowers as dictated by the multi-objective function (S_{cross}). The resulting optimized hybrid seeds are subsequently harvested, completing the transformation of the genetic compatibility predictions into a physical agricultural product.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A system for genetic profile analysis and compatibility assessment, comprising:
(a) a specialized hardware acceleration unit comprising field-programmable gate arrays (FPGAs) configured to implement sequence alignment algorithms to process genetic sequence data;
(b) a genetic profile analyzer comprising:
(i) a data acquisition module configured to receive genetic sequence data;
(ii) a hardware-accelerated sequence alignment processor configured to identify genetic markers within the received genetic sequence data using parallel processing architecture;
(iii) a trait analyzer configured to classify identified genetic markers as dominant or recessive traits based on predefined genetic inheritance models;
(c) a genetic compatibility assessment engine comprising:
(i) a match scoring module configured to calculate genetic compatibility scores using weighted comparison of identified dominant and recessive traits;
(ii) a risk assessment module configured to evaluate genetic compatibility risks using statistical probability models;
(d) a database management system configured to:
(i) store genetic profile data using data structures optimized for genetic sequence retrieval;
(ii) maintain genetic marker indices optimized for accelerated data retrieval;
wherein the system implements a defined processing pipeline for transferring genetic data between modules and utilizes the field-programmable gate arrays (FPGAs) to accelerate genetic sequence alignment operations through parallel computation of sequence matching matrices.

2. The system of claim 1, wherein the genetic compatibility assessment engine is further configured to:
(a) identify complementary genetic profiles by computing predicted offspring genotype probability distributions at a plurality of loci based on parental genotype data, and evaluating whether the predicted offspring genotype distributions indicate favorable trait expression;
(b) calculate an optimal mating distance score that balances genetic diversity benefits against incompatibility risks; and
(c) generate a ranked list of genetic matches based on the calculated genetic compatibility scores and optimal mating distance scores.

3. The system of claim 1, wherein the trait analyzer is further configured to:
(a) classify genetic markers according to inheritance patterns including dominant, recessive, co-dominant, and polygenic traits;
(b) identify carrier status for recessive disease alleles; and
(c) evaluate immune system compatibility markers including Human Leukocyte Antigen (HLA) genes in humans or corresponding markers in non-human species.

4. The system of claim 1, wherein the field-programmable gate arrays (FPGAs) are configured to:
(a) implement an array architecture specifically optimized for executing Smith-Waterman or Needleman-Wunsch sequence alignment algorithms;
(b) incorporate dedicated logic blocks for parallel computation of nucleotide scoring matrices; and
(c) utilize specialized memory access patterns that minimize data transfer latency during genetic sequence comparison operations.

5. The system of claim 1, wherein the system is configured to operate across multiple species through:
(a) a layer of abstraction that translates species-specific genetic concepts into standardized representations;
(b) species-specific modules that address unique genetic characteristics of humans, animals, or plants; and
(c) cross-species orthologous gene mapping to facilitate knowledge transfer across application domains.

6. The system of claim 5, wherein:
(a) for human applications, the species-specific module is configured to evaluate compatibility at Human Leukocyte Antigen (HLA) loci, screen for carrier status of monogenic recessive disorders, and compute polygenic risk scores for hereditary disease;
(b) for animal applications, the species-specific module is configured to compute genomic estimated breeding values (GEBVs) for production, health, and fertility traits, evaluate deleterious recessive haplotype carrier status, and generate cohort-level mating assignments subject to herd management constraints; and
(c) for plant applications, the species-specific module is configured to evaluate loci associated with yield components, disease resistance gene series, and environmental adaptability, and generate cross-pollination plans optimized for heterosis while controlling for deleterious allele fixation.

7. A method for genetic profile analysis and compatibility assessment executed by a computing device communicatively coupled to a hardware acceleration unit, comprising:
(a) receiving genetic sequence data from multiple subjects;
(b) processing the genetic sequence data using field-programmable gate arrays (FPGAs) configured to accelerate sequence alignment operations through parallel computation;
(c) identifying genetic markers within the processed genetic sequence data;
(d) classifying the identified genetic markers as dominant or recessive traits based on predefined genetic inheritance models;
(e) calculating genetic compatibility scores between subjects using weighted comparison of the classified genetic markers;
(f) evaluating genetic compatibility risks using statistical probability models; and
(g) generating a ranked list of genetically compatible matches based on the calculated compatibility scores and evaluated risks.

8. The method of claim 7, further comprising:
(a) analyzing DNA matching patterns to identify complementary genetic profiles that would function optimally together in offspring;
(b) calculating an optimal mating distance that balances genetic diversity benefits with incompatibility risks; and
(c) predicting offspring trait expressions using statistical probability models based on parental genetic markers.

9. The method of claim 7, wherein calculating genetic compatibility scores comprises:
(a) assigning different weights to genetic markers based on their impact on traits of interest;
(b) evaluating compatibility at critical genetic loci including immune system genes;
(c) calculating a genetic distance measure between subjects; and
(d) integrating these factors into a composite compatibility score.

10. The method of claim 7, wherein processing the genetic sequence data using field-programmable gate arrays (FPGAs) comprises:
(a) implementing hardware-optimized versions of sequence alignment algorithms;
(b) utilizing an array architecture for parallel computation of alignment matrices; and
(c) employing specialized memory access patterns that optimize cache utilization and minimize memory bandwidth requirements.

11. A method for evaluating genetic compatibility between potential mates, comprising:
(a) obtaining genetic profiles from potential mates through DNA sequencing;
(b) analyzing the genetic profiles using hardware-accelerated sequence alignment to identify genetic markers;
(c) classifying the genetic markers according to inheritance patterns and associated traits;
(d) screening for recessive disease mutations and calculating carrier risk probabilities;
(e) calculating genetic compatibility scores between a subject and potential mates using weighted comparison of genetic markers;
(f) determining an optimal genetic distance between potential mates that balances heterosis benefits with genetic incompatibility risks; and
(g) generating a prioritized list of genetically compatible mates using hardware-accelerated sequence alignment based on the calculated scores, recessive mutation screening, and optimal genetic distance.

12. The method of claim 11, further comprising:
(a) evaluating compatibility at specific genetic loci, including Human Leukocyte Antigen (HLA) genes or equivalent immune system markers;
(b) calculating statistical probabilities for offspring trait expression based on potential mate combinations;
(c) predicting the likelihood of hereditary disease transmission in offspring; and
(d) generating detailed compatibility reports that present findings in an actionable format.

13. The method of claim 7, wherein evaluating genetic compatibility risks using statistical probability models comprises:
(a) estimating posterior distributions for trait parameters and marker effects using Bayesian inference algorithms;
(b) computing an offspring genotype probability distribution for candidate pairings; and
(c) propagating parental genotype uncertainty and the posterior distributions into predicted offspring outcome distributions utilizing Monte Carlo simulations.

14. The method of claim 13, wherein calculating genetic compatibility scores further comprises computing a complementary index that mathematically integrates an expected economic or trait value with penalty terms for predicted dystocia risk, stillbirth risk, or probability of deleterious homozygosity derived from the Monte Carlo simulations.

15. The method of claim 7, further comprising generating a cohort-level mating assignment plan by optimizing an objective function across a plurality of candidate pairings utilizing combinatorial optimization techniques, subject to predefined herd management or seed production constraints, and physically breeding the corresponding cohorts according to the assignment plan.

16. The method of claim 7, wherein identifying genetic markers within the processed genetic sequence data further comprises identifying epigenetic markers, including DNA methylation patterns and histone modifications, and wherein evaluating genetic compatibility risks comprises predicting epigenetic compatibility and environmentally-induced epimutation transmission.

17. A non-transitory computer-readable medium storing instructions that, when executed by a processor, cause the processor to perform a method for genetic profile analysis and compatibility assessment, the method comprising:
(a) receiving genetic sequence data from multiple subjects;
(b) processing the genetic sequence data using hardware-accelerated sequence alignment operations;
(c) identifying genetic markers within the processed genetic sequence data;
(d) classifying the identified genetic markers as dominant or recessive traits;
(e) calculating genetic compatibility scores between subjects using weighted comparison of the classified genetic markers;
(f) evaluating genetic compatibility risks using statistical probability models; and
(g) generating a ranked list of genetically compatible matches based on the calculated compatibility scores and evaluated risks.

18. The system of claim 1, wherein the system evaluates genetic markers at a plurality of loci numbering at least 3,000 markers for livestock breeding applications or at least 1,000 markers for human fertility applications.

19. The method of claim 13, wherein estimating posterior distributions for trait parameters and marker effects comprises employing a Bayesian regression method selected from the group consisting of BayesA, BayesB, BayesC, BayesCπ, and Bayesian LASSO.

20. The method of claim 13, wherein estimating posterior distributions for trait parameters and marker effects comprises implementing a hierarchical Bayesian model structure comprising:
(i) a first level estimating effects of subject genetic markers on phenotypic outcomes;
(ii) a second level governing the distribution of marker effects across the genome through hyperparameters; and
(iii) a third level modeling cross-trait correlations through multivariate Bayesian methods.

21. The method of claim 13, wherein propagating parental genotype uncertainty and the posterior distributions into predicted offspring outcome distributions further comprises employing one or more alternative uncertainty propagation methods selected from: quasi-Monte Carlo methods utilizing low-discrepancy sequences, importance sampling, sequential Monte Carlo methods, and polynomial chaos expansion.
